(19) United States
(12) Patent Application Publication
(10) Pub. No.: US 2017/0231481 A1
(43) Pub. Date: Aug. 17, 2017

(54) INSERTION DEVICE ASSEMBLY FOR NASAL SINUSES

(71) Applicant: OLYMPUS CORPORATION, Tokyo (JP)

(72) Inventor: Hiroyuki KOJO, Hachioji-shi (JP)

(73) Assignee: OLYMPUS CORPORATION, Tokyo (JP)

(21) Appl. No.: 15/042,284

(22) Filed: Feb. 12, 2016

(51) Int. Cl. A61B 1/233; A61B 1/07; A61B 1/015; A61B 1/00; A61B 1/005

(52) U.S. Cl. CPC A61B 1/233; A61B 1/00154; A61B 1/00135; A61B 1/005; A61B 1/015; A61B 1/07; A61B 1/0014

(57) ABSTRACT

An insertion device assembly for nasal sinuses includes: a guide pipe bent portion on the distal side of the guide pipe main body; a guide pipe distal opening on the distal side of the bent portion; a sheath inserted into the bent portion and the guide pipe distal opening from a proximal side opening of the main body through the main body to be movable back and forth; a sheath distal opening at the distal side of the sheath; an insertion portion of an endoscope inserted through the sheath to be movable back and forth; and an endoscope operation portion passing the sheath distal opening and the distal end of the insertion portion of the endoscope through the bent portion and protruding the distal end of the insertion portion of the endoscope from the guide pipe distal opening.

### BACKGROUND OF THE INVENTION

1. Field of the Invention

This invention relates to an insertion device assembly for nasal sinuses.

2. Description of the Related Art

For example, U.S. Pat. No. 7,559,925 has disclosed a treatment instrument in which a light guide fiber is inserted through a guide pipe. The treatment instrument guides the light guide fiber to a paranasal sinus while visually recognizing, through the skin and bone of a patient, light emitted from the distal end of the light guide fiber. That is, the position of the distal end of the light guide fiber in the nasal cavity is estimated on the basis of the light emitted from the distal end of the light guide fiber.

### BRIEF SUMMARY OF THE INVENTION

According to one embodiment of the present invention, an insertion device assembly for nasal sinuses includes: a guide pipe main body; a guide pipe bent portion provided on the distal side of the guide pipe main body; a guide pipe distal opening provided on the distal side of the guide pipe bent portion; a sheath which is inserted into the guide pipe bent portion and the guide pipe distal opening from a proximal side opening of the guide pipe main body through the guide pipe main body to be movable back and forth; a sheath distal opening provided at the distal side of the sheath; an insertion portion of an endoscope which is inserted through the sheath to be movable back and forth; and an endoscope operation portion which is configured to pass the sheath distal opening and the distal end of the insertion portion of the endoscope through the guide pipe bent portion and which is configured to protrude the distal end of the insertion portion of the endoscope from the guide pipe distal opening.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1A is a schematic diagram of a treatment system according to a first embodiment;

FIG. 1B is a schematic front view showing an insertion device assembly of the treatment system viewed from the direction of an arrow 1B in FIG. 1A;

FIG. 2A is a schematic top view showing the insertion device assembly of the treatment system viewed from the direction of an arrow 2A in FIG. 1A;

FIG. 2B is a schematic longitudinal sectional view of the insertion device assembly along the arrow 2B-2B line in FIG. 2A;

FIG. 3 is a schematic longitudinal sectional view showing the distal end of an insertion portion of an endoscope of the insertion device assembly of the treatment system according to the first embodiment;

FIG. 4A is a schematic cross sectional view of an insertion device assembly along the arrow line 4A-4A in FIG. 2B;

FIG. 4B is a schematic cross sectional view of the insertion device assembly along the arrow line 4B-4B in FIG. 2B;

FIG. 5A is a schematic diagram showing, in enlarged form, the vicinity of a connecting pipe of the insertion device assembly at a position indicated by a sign 5A in FIG. 2B;

FIG. 5B is a schematic diagram showing, in enlarged form, the vicinity of a first operation element of the insertion device assembly at a position indicated by a sign 5B in FIG. 2B;

FIG. 6 is a schematic longitudinal sectional view showing a sheath of the insertion device assembly of the treatment system according to the first embodiment;

FIG. 7 is a schematic longitudinal sectional view showing a guide pipe of the insertion device assembly of the treatment system according to the first embodiment;

FIG. 8A is a schematic diagram showing how the distal end of the sheath and the distal end of the insertion portion of the endoscope are brought into abutment with the proximal side surface of a bent portion of a bent pipe of the guide pipe of the insertion device assembly of the treatment system according to the first embodiment by the use of an interlock portion so that the distal end of the sheath and the distal end of the insertion portion of the endoscope face a guide portion which is continuously formed in the bent portion;

FIG. 8B is a schematic diagram showing how the distal end of the sheath and the distal end of the insertion portion of the endoscope pass through the bent portion of the bent pipe of the guide pipe of the insertion device assembly of the treatment system according to the first embodiment by the use of the interlock portion so that the distal end of the sheath and the distal end of the insertion portion of the endoscope are located between the bent portion and the distal end of the guide pipe;

FIG. 8C is a schematic diagram showing how the interlock portion is detached from the first and second operation elements after the distal end of the sheath and the distal end of the insertion portion of the endoscope have passed through the bent portion of the bent pipe of the guide pipe of the insertion device assembly of the treatment system according to the first embodiment;

FIG. 8D is a schematic diagram showing how the distal end of the insertion portion of the endoscope protrudes from the distal end of the guide pipe while the position of the sheath relative to the guide pipe is maintained after the distal end of the sheath and the distal end of the insertion portion of the endoscope have passed through the bent portion of the bent pipe of the guide pipe of the insertion device assembly of the treatment system according to the first embodiment and then the interlock portion has been detached from the first and second operation elements;

FIG. 9A is a schematic diagram showing the positional relation of a maxillary sinus of nasal sinuses to the nose, and showing how the distal end of the guide pipe of the insertion device assembly is located at the entrance of the maxillary sinus so that the distal end of the sheath and/or the distal end of the insertion portion of the endoscope protrude/protrudes from the distal end of the guide pipe;

FIG. 9B is a schematic sectional view showing the positional relation of the maxillary sinus of the nasal sinuses to the nose, and showing how the distal end of the guide pipe of the insertion device assembly is located at the entrance of the maxillary sinus so that the distal end of the sheath and/or the distal end of the insertion portion of the endoscope protrude/protrudes from the distal end of the guide pipe;

FIG. 10A is a schematic diagram showing how the distal end of the sheath passes through the bent portion of the bent pipe of the guide pipe in the insertion device assembly of the treatment system according to the first embodiment without the use of the interlock portion, and the distal end of the insertion portion of the endoscope located in a straight pipe;

FIG. 10B is a schematic diagram showing how the resilience of the sheath is received when the distal end of the insertion portion of the endoscope is to pass through the bent portion of the guide pipe from the inside of the straight pipe while the distal end of the sheath passes through the bent portion of the bent pipe of the guide pipe without the use of the interlock portion in the insertion device assembly of the treatment system according to the first embodiment;

FIG. 11 is a schematic diagram showing how the distal end of the sheath and the distal end of the insertion portion of the endoscope pass through the bent portion of the bent pipe of the guide pipe of the insertion device assembly of the treatment system according to a modification of the first embodiment by the use of the interlock portion so that the distal end of the sheath and the distal end of the insertion portion of the endoscope are located between the bent portion and the distal end of the guide pipe, and showing how the distal end of the sheath is located between the distal end and proximal end of a distal hard portion of the insertion portion of the endoscope;

FIG. 12A is a schematic diagram showing how the distal end of the sheath and the distal end of the insertion portion of the endoscope pass through the bent portion of the bent pipe of the guide pipe of the insertion device assembly of the treatment system according to the modification of the first embodiment by the use of the interlock portion so that the distal end of the sheath and the distal end of the insertion portion of the endoscope are located between the bent portion and the distal end of the guide pipe, and showing a state immediately before a click mechanism adds a click feeling;

FIG. 12B is a schematic diagram showing how the distal end of the sheath and the distal end of the insertion portion of the endoscope pass through the bent portion of the bent pipe of the guide pipe of the insertion device assembly of the treatment system according to the modification of the first embodiment by the use of the interlock portion so that the distal end of the sheath and the distal end of the insertion portion of the endoscope are located between the bent portion and the distal end of the guide pipe, and showing a state immediately after the click mechanism has added the click feeling;

FIG. 13A is a schematic diagram showing how the distal end of the sheath and the distal end of the insertion portion of the endoscope pass through the bent portion of the bent pipe of the guide pipe of the insertion device assembly of the treatment system according to a second embodiment by the use of the interlock portion so that the distal end of the sheath and the distal end of the insertion portion of the endoscope are located between the bent portion and the distal end of the guide pipe;

FIG. 13B is a schematic diagram showing how the interlock state of the interlock portion is canceled into a non-interlock state so that the distal end of the insertion portion of the endoscope protrudes from the distal end of the sheath after the distal end of the sheath and the distal end of the insertion portion of the endoscope have passed through the bent portion of the bent pipe of the guide pipe of the insertion device assembly of the treatment system according to the second embodiment;

FIG. 14A is a schematic diagram showing how the distal end of the sheath and the distal end of the insertion portion of the endoscope pass through the bent portion of the bent pipe of the guide pipe of the insertion device assembly of the treatment system according to a first modification of the second embodiment by the use of the interlock portion so that the distal end of the sheath and the distal end of the insertion portion of the endoscope are located between the bent portion and the distal end of the guide pipe;

FIG. 14B is a schematic diagram showing how the interlock state of the interlock portion is canceled into the non-interlock state so that the distal end of the insertion portion of the endoscope protrudes from the distal end of the sheath after the distal end of the sheath and the distal end of the insertion portion of the endoscope have passed through the bent portion of the bent pipe of the guide pipe of the insertion device assembly of the treatment system according to the first modification of the second embodiment;

FIG. 15A is a schematic diagram showing how the distal end of the sheath and the distal end of the insertion portion of the endoscope pass through the bent portion of the bent pipe of the guide pipe of the insertion device assembly of the treatment system according to a second modification of the second embodiment by the use of the interlock portion so that the distal end of the sheath and the distal end of the insertion portion of the endoscope are located between the bent portion and the distal end of the guide pipe;

FIG. 15B is a schematic diagram showing how the interlock state of the interlock portion is canceled into the non-interlock state so that the distal end of the insertion portion of the endoscope protrudes from the distal end of the sheath after the distal end of the sheath and the distal end of the insertion portion of the endoscope have passed through the bent portion of the bent pipe of the guide pipe of the insertion device assembly of the treatment system according to the second modification of the second embodiment;

FIG. 16A is a schematic diagram showing how the sheath is interlocked to operate an operation element of the endoscope so that the distal end of the sheath and the distal end of the insertion portion of the endoscope pass through the bent portion of the bent pipe of the guide pipe of the insertion device assembly of the treatment system according to a third embodiment, and that the distal end of the sheath and the distal end of the insertion portion of the endoscope are located between the bent portion and the distal end of the guide pipe;

FIG. 16B is a schematic diagram showing how the sheath is interlocked to operate the operation element of the endoscope so that the distal end of the sheath and the distal end of the insertion portion of the endoscope pass through the bent portion of the bent pipe of the guide pipe of the insertion device assembly of the treatment system according to the third embodiment, and then the position of the distal end of the insertion portion of the endoscope relative to the guide pipe is maintained to pull the sheath from the distal side of the guide pipe; and

FIG. 16C is a schematic diagram showing how the sheath is interlocked to operate the operation element of the endoscope so that the distal end of the sheath and the distal end of the insertion portion of the endoscope pass through the bent portion of the bent pipe of the guide pipe of the insertion device assembly of the treatment system according to the third embodiment, and the position of the distal end of the insertion portion of the endoscope relative to the guide pipe is maintained to pull the sheath from the distal side of the guide pipe, and then the operation element is operated to protrude the distal end of the insertion portion of the endoscope from the distal side of the guide pipe.

### DETAILED DESCRIPTION OF THE INVENTION

Hereinafter, embodiments of this invention will be described with reference to the drawings.

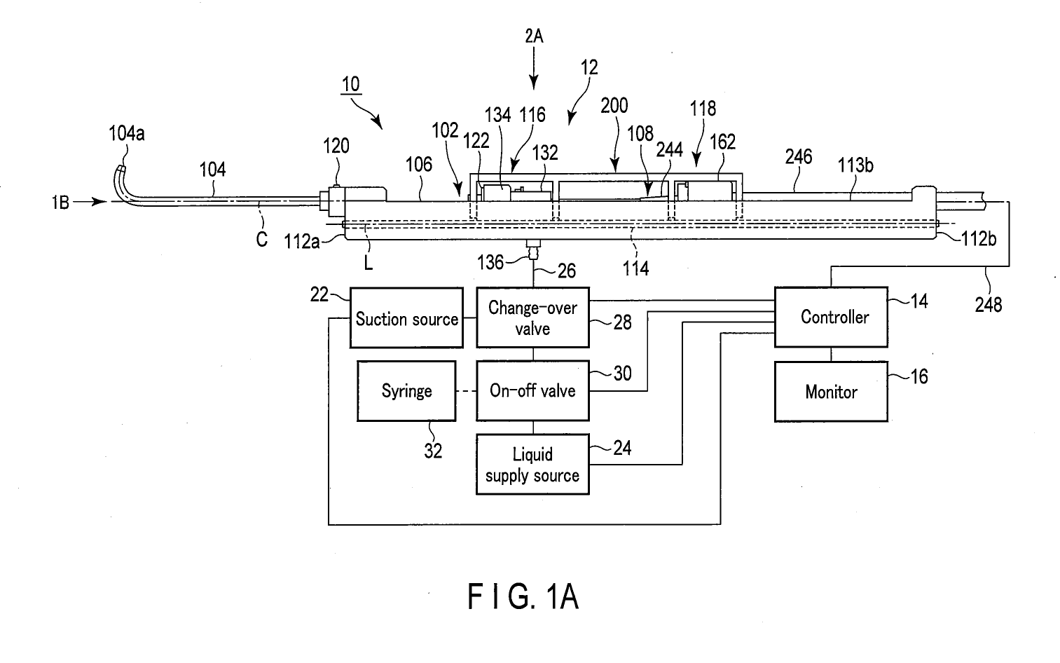

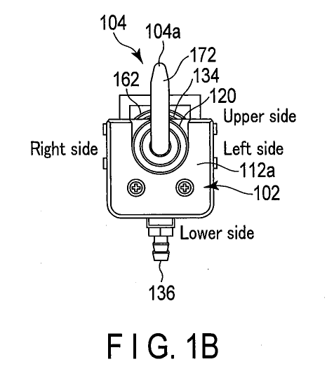

The first embodiment is described with reference to FIG. 1A to FIG. 10B.

As shown in FIG. 1A, a treatment system (endoscopic system) 10 according to this embodiment includes an insertion device assembly 12 for a nasal sinuses, a controller 14, and a monitor 16.

A suction source 22 and a liquid supply source (liquid sending source) 24 are connectable to the insertion device assembly 12. A change-over valve 28 such as a three-way cock is provided between the end of a tube 26 extending from the insertion device assembly 12, the suction source 22, and the liquid supply source 24. Thus, a user can selectively use the suction source 22 and the liquid supply source 24 for the insertion device assembly 12 by operating the change-over valve 28. It is also preferable that an on-off valve 30 such as a three-way cock to which a syringe 32 can be removably connected is provided for medication, for example, between the change-over valve 28 and the liquid supply source 24. The change-over valve 28 and the on-off valve 30 may be electromagnetically operated by turning an unshown switch connected to the controller 14 or may be manually switched.

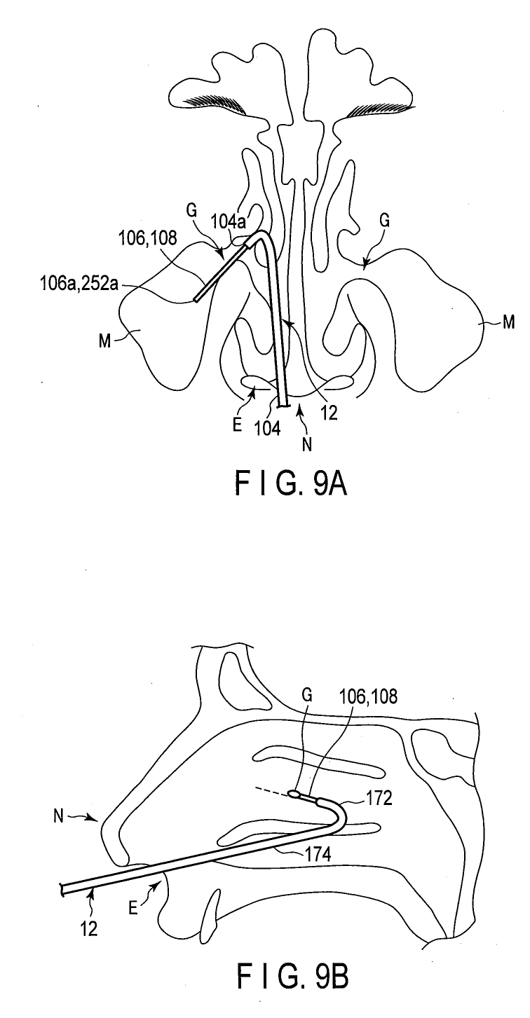

A liquid supplied from the liquid supply source 24 can be suitably selected. The liquid supply source 24 can supply a physiological saline to clean an affected part in, for example, a maxillary sinus M of nasal sinuses inside a nose N (see FIG. 9A and FIG. 9B). Moreover, the liquid supply source 24 can supply a chemical to treat the affected part. As the chemical, steroid or an antibacterial agent is mainly administered. Here, instead of simply administering the chemical, temperature-responding gel which increases viscosity at about the body temperature may be used to extend the time in which the chemical remains in the affected part. In this case, if the chemical is administered to the affected part, the viscosity of the chemical increases due to the body temperature of a patient, so that the chemical does not easily flow out of the affected part and remains for a longer time. That is, if such a chemical is administered to the affected part, the chemical is easily retained in the affected part. Viscous matter present in, for example, the maxillary sinus M of the nasal sinuses and around the affected part inside a nasal cavity can be removed by actuating the suction source 22. When the affected part and the parts therearound are cleaned with the physiological saline, this cleaning liquid can be removed together with the viscous matter.

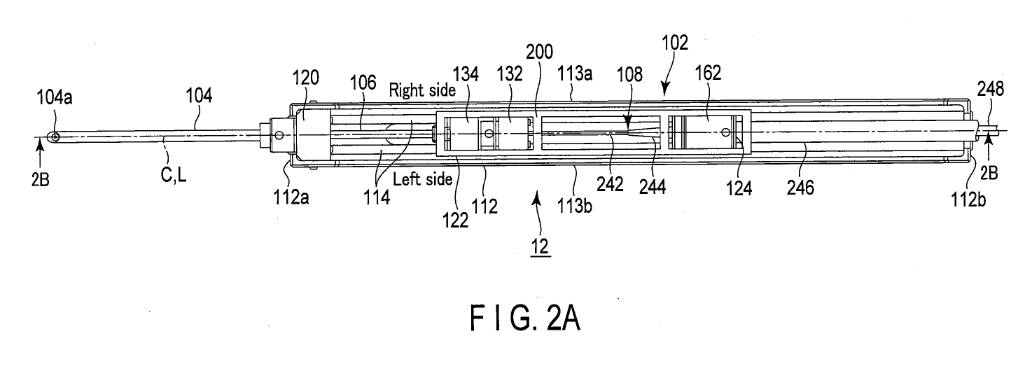

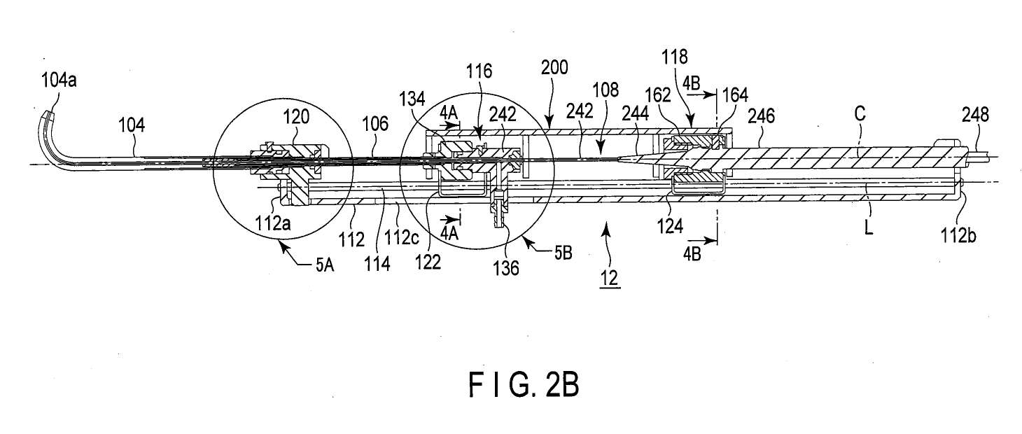

As shown in FIG. 2A and FIG. 2B, the insertion device assembly 12 includes a handle unit 102, a guide pipe 104, a sheath 106, and an endoscope 108.

The endoscope 108 includes an insertion portion 242 which is inserted through the sheath 106 to be movable back and forth, an antibreak 244, a support portion 246, and a cable 248. The insertion portion 242 has a length of, for example, about 200 mm, and is formed to be able to protrude, for example, about 100 mm from a later-described distal end (distal opening) 104a of the guide pipe 104. The insertion portion 242 preferably has a small outside diameter of about 1 mm to 2 mm, and particularly preferably has an outside diameter of about 1.4 mm. The outside diameter of the insertion portion 242 is extremely small, an endoscope 108 of any type such as a fiber type or an image pickup device type including a CCD or a CMOS may be used, but the endoscope 108 of a scan type is preferably used. By the use of such an endoscope 108, the diameter of the insertion portion 242 can be small, and a satisfactory image quality can be obtained.

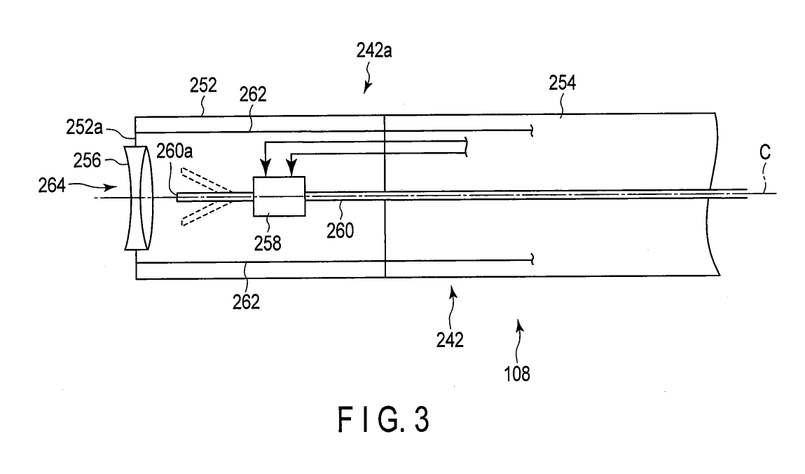

Although the endoscope 108 is publicly known and is therefore not described in detail, the internal structure of distal end 242a of the insertion portion 242 is formed as shown in FIG. 3.

As shown in FIG. 3, the insertion portion 242 of the scanning endoscope 108 includes a distal hard portion 252, a flexible pipe 254, an illumination window 256, an actuator 258, an illumination fiber 260, and light receiving fibers 262. Among these components, the illumination window 256, the actuator 258, the illumination fiber 260, and the light receiving fibers 262 constitute an observation optical system 264. That is, the observation optical system 264 is provided inside the insertion portion 242. In the observation optical system 264, the actuator 258, the illumination fiber 260, and the light receiving fibers 262 are optically and/or electrically connected to the controller 14 shown in FIG. 1A.

The controller 14 shown in FIG. 1A controls the observation optical system 264 of the endoscope 108. The controller 14 controls the operation of the actuator 258. The controller 14 has an unshown light source of, for example, white light, and properly causes light for observation to enter the illumination fiber 260. The controller 14 images the light received by the light receiving fibers 262.

The distal hard portion 252, the illumination window 256, the actuator 258, the distal end of the illumination fiber 260, and the distal ends of the light receiving fibers 262 are provided at the distal end 242a of the insertion portion 242.

The illumination window 256 and the distal ends of the light receiving fibers 262 are fixed to a distal end face (distal end) 252a of the distal hard portion 252. The distal ends of the light receiving fibers 262 are fixed around the illumination window 256 at proper intervals.

The distal hard portion 252 is provided with the actuator 258 on the proximal side of the illumination window 256. The actuator 258 supports the distal end (a part closer to the proximal side than the most distal end) of the illumination fiber 260. The actuator 258 is shaken by the controller 14, for example, in a spiral manner. Thus, a distal end 260a of the illumination fiber 260 is spirally shaken in accordance with the operation of the actuator 258. Therefore, the surface of the subject is spirally scanned with the illumination light through the distal end of the illumination fiber 260 and the illumination window 256. The light receiving fibers 262 receive reflected light from the subject, and guide the light to the controller 14. The controller 14 shown in FIG. 1A images the light received by the light receiving fibers 262, and displays the formed image on the monitor 16 connected to the controller 14.

As shown in FIG. 3, on the proximal side of the distal hard portion 252, the flexible pipe 254 extends to this proximal side. The length of the distal hard portion 252 is, for example, about 10 mm. Thus, the flexible pipe 254 accounts for the most of the total length of the insertion portion 242. In other words, most of the insertion portion 242 is formed as a flexible part. The antibreak 244 is fixed to the proximal end of the flexible pipe 254. The support portion 246 is fixed to the proximal end of the antibreak 244. The cable 248 is fixed to the proximal end of the support portion 246. The proximal end of the cable 248 is connected to the controller 14.

The handle unit 102 is grasped by the user and suitably operated. In this endoscopic treatment system 10, the insertion portion 242 of the endoscope 108, the sheath 106, and the guide pipe 104 are arranged in order from the inside of the central axis C to the outside.

The handle unit 102 includes a main body 112, guide rails 114, a first operation element (sheath operation portion) 116 which moves the sheath 106, and a second operation element (endoscope operation portion) 118 which supports and moves the insertion portion 242 of the endoscope 108.

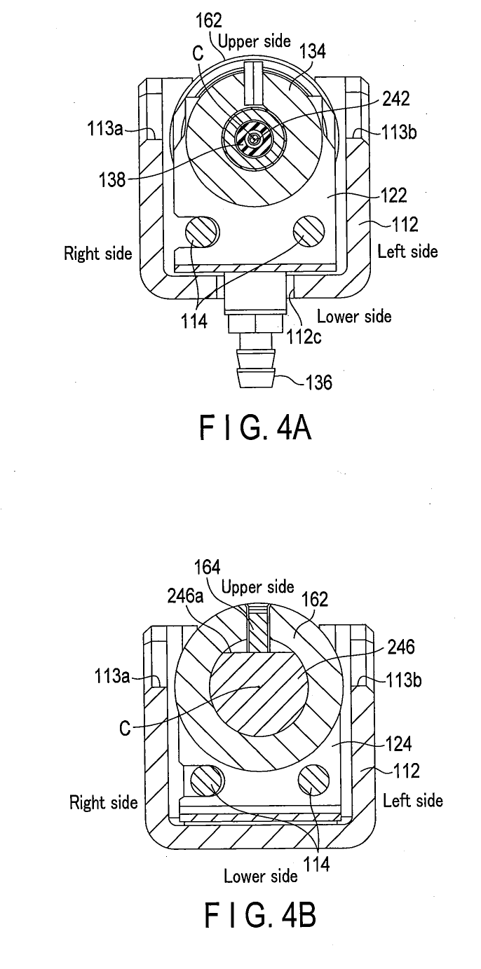

As shown in FIG. 1A to FIG. 2B, the main body 112 defines a longitudinal axis L by its distal end 112a and proximal end 112b. As shown in FIG. 4A and FIG. 4B, the cross section of the main body 112 is substantially U-shaped. The upper side of the main body 112 is open. As shown in FIG. 2A and FIG. 2B, the guide rails 114 are fixed to the distal end 112a and the proximal end 112b of the main body 112, respectively.

The guide rail 114 is formed by a rod or a pipe which couples the distal end 112a and the proximal end 112b of the main body 112 straight to each other. The guide rail 114 is made of a rigid material such as stainless steel. One guide rail 114 is sufficient, but two (a pair of) guide rails 114 are preferably formed parallel to each other.

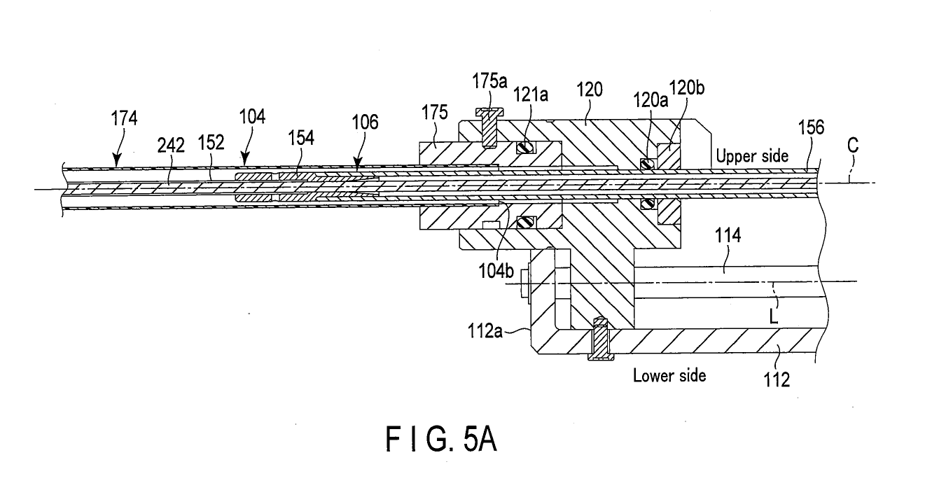

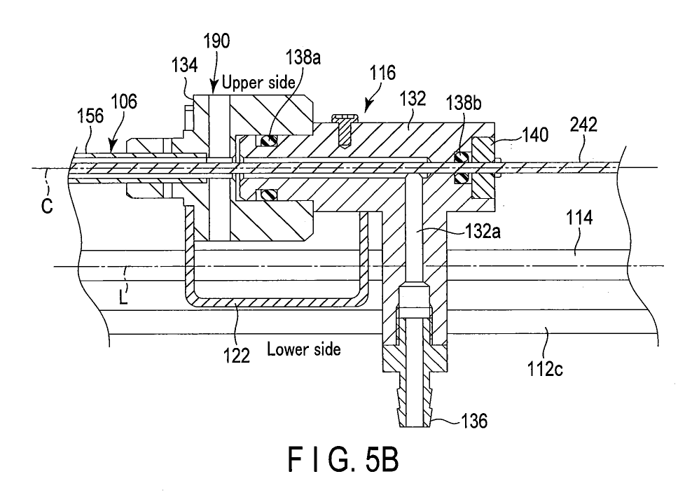

As shown in FIG. 2A, FIG. 2B, and FIG. 5A, a connection pipe 120 to which the proximal end of the guide pipe 104 is coupled is fixed to the distal end 112a of the main body 112. An O-ring 120a is provided between the inner circumferential surface of the connection pipe 120 and the outer circumferential surface of a later-described inner pipe 156 of the sheath 106. The O-ring 120a exerts suitable frictional force between the inner circumferential surface of the O-ring 120a and the outer circumferential surface of the inner pipe 156 of the sheath 106. Thus, the sheath 106 can be turned around the central axis C by the user operation and can be moved along the central axis C, but is inhibited from free movement. The O-ring 120a is prevented by a holding member 120b from coming off the proximal side of the connection pipe 120.

As shown in FIG. 2A, FIG. 2B, FIG. 4A, FIG. 4B, FIG. 5A, and FIG. 5B, first and second holders 122 and 124 which move along the guide rail 114 are provided between the distal end 112a and the proximal end 112b of the main body 112 of the handle unit 102. The first holder 122 is close to the distal end 112a of the main body 112, and the second holder 124 is close to the proximal end 112b of the main body 112. The first and second holders 122 and 124 can come closer to and come in and out of contact with each other along the guide rail 114.

As shown in FIG. 4A and FIG. 5B, a T-shaped pipe 132 which is in communication with the space between the outer circumferential surface of the insertion portion 242 of the endoscope 108 and the inner circumferential surface of the sheath 106 is supported on the first holder 122. A first rotor 134 rotatable around the central axis C is provided at the distal end of the T-shaped pipe 132. The first holder 122, the T-shaped pipe 132, and the first rotor 134 constitute the first operation element 116. The first operation element 116 can move the sheath 106 in its axial direction relative to the guide pipe 104 and the insertion portion 242 and can also turn the sheath 106 around its axis. That is, the first operation element 116 can move the sheath 106 relative to the guide pipe 104 and the insertion portion 242.

A joint 136 which communicates with the central axis C of the T-shaped pipe 132 and the first rotor 134 through a pipeline 132a is connected to the T-shaped pipe 132. The joint 136 protrudes from an opening 112c of the main body 112 of the handle unit 102. The suction source 22, the liquid supply source 24, the change-over valve 28, and the on-off valve 30 are connected to the joint 136 as shown in FIG. 1A.

O-rings 138a and 138b are respectively disposed between the outer circumferential surface of the distal end of the T-shaped pipe 132 and the first rotor 134 and between the inner circumferential surface of the proximal end of the T-shaped pipe 132 and the outer circumferential surface of the insertion portion 242 of the endoscope 108. In particular, the O-ring 138b is prevented by a holding member 140 from coming off the proximal side of the T-shaped pipe 132. Thus, when a gas or a liquid is supplied from the joint 136, the gas or the liquid can be guided toward the distal side of the T-shaped pipe 132.

The O-ring 138b exerts suitable frictional force between its inner circumferential surface and the outer circumferential surface of the insertion portion 242 of the endoscope 108. Thus, the insertion portion 242 of the endoscope 108 can be turned around the central axis C by the user operation and can be moved along the central axis C, but is inhibited from free movement. Here, the frictional force between the inner circumferential surface of the O-ring 138b and the outer circumferential surface of the insertion portion 242 of the endoscope 108 is set to such a degree that the second operation element 118 does not unintentionally move in response to the movement of the first operation element 116 and the movement of the insertion portion 242.

The O-ring 138a exerts suitable frictional force between its outer circumferential surface and the first rotor 134. Thus, the first rotor 134 can be turned around the central axis C by the user operation and can be moved along the central axis C, but is inhibited from free movement.

Although the first holder 122 has the first rotor 134 in the example described here, the mechanism which rotates the sheath 106 is not always needed.

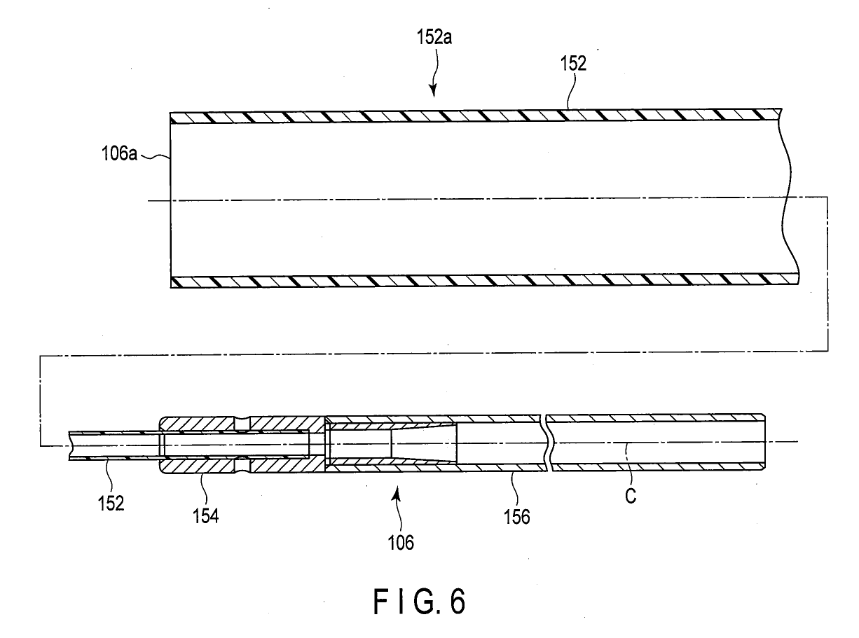

Here, the sheath 106 shown in FIG. 6 has an inside diameter which allows the insertion portion 242 to be inserted therethrough so that the distal end 252a of the insertion portion 242 can protrude from a sheath distal opening (distal end) 106a provided at the distal end of the sheath 106. The sheath 106 is inserted into a bent pipe 172 and the distal opening 104a from a proximal side opening 106b of the guide pipe 104 through a straight pipe 174 of the guide pipe 104 to be movable back and forth. Thus, the sheath 106 is inserted through the guide pipe 104 so that its distal end 106a can protrude from the distal end 104a provided on the distal side of a later-described bent portion 172c of the guide pipe 104.

As shown in FIG. 6, the sheath 106 has a sheath body 152, a sheath holder 154, and an inner pipe in order from the distal side to the proximal side.

The sheath body 152 is formed into a tubular shape by an elastically deformable resinous material having a thickness of, for example, about 0.1 mm. The sheath body 152 is filled with, for example, a braided tube (not shown) called a braid. Thus, the sheath body 152 according to this embodiment is formed more firmly than when simply formed by the resinous material. That is, the sheath body 152 of the sheath 106 is thin, but has high performance of tracking the rotation around the central axis C, is easily bendable, and is formed to be unbreakable so that a hollow part is kept therein. The sheath body 152 is preferably formed to be more unbendable than the flexible pipe 254 of the insertion portion 242 of the endoscope 108 shown in FIG. 3. Thus, when the whole distal hard portion 252 of the insertion portion 242 of the endoscope 108 and part of the flexible pipe 254 protrude from the distal end 106a of the sheath body 152, the position of the distal hard portion 252 of the insertion portion 242 of the endoscope 108 can be kept in a desired state.

The sheath holder 154 shown in FIG. 5A and FIG. 6 are cylindrically formed by a rigid material such as stainless steel. The outer circumferential surface of the proximal end of the sheath body 152 is fixed to the inner circumferential surface of the sheath holder 154 by, for example, adhesive bonding. The inner circumferential surface of the distal end of the inner pipe 156 made of a rigid material such as stainless steel is fixed to the outer circumferential surface of the sheath holder 154 by, for example, adhesive bonding. The proximal end of the inner pipe 156 is fixed to the inner circumferential surface of the first rotor 134 by, for example, adhesive bonding. Thus, the inner pipe 156, the sheath holder 154, and the sheath body 152, that is, the sheath 106 moves together with the movement of the first operation element 116.

More specifically, if the first operation element 116 is moved forward relative to the handle unit 102 along the central axis C, the inner pipe 156, the sheath holder 154, and the sheath body 152, that is, the sheath 106 moves forward along the central axis C. If the first operation element 116 is moved backward relative to the handle unit 102 along the central axis C, the inner pipe 156, the sheath holder 154, and the sheath body 152, that is, the sheath 106 moves backward along the central axis C. If the first operation element 116 is turned or rotated relative to the handle unit 102 around the central axis C, the inner pipe 156, the sheath holder 154, and the sheath body 152, that is, the sheath 106 turns or rotates around the central axis C in the same direction as the turning or rotation direction of the first operation element 116.

As shown in FIG. 2A, FIG. 2B, and FIG. 4B, the second holder 124 is provided with a second rotor 162 which is rotatable around the central axis C. The second rotor 162 supports the support portion 246 on the proximal side of the antibreak 244 of the insertion portion 242 of the endoscope 108.

Here, the cross section of the support portion 246 of the endoscope 108 is substantially D-shaped. That is, the support portion 246 has a plane 246a. A pin 164 prevents the plane 246a of the support portion 246 from rotating relative to the second rotor 162. Thus, if the second rotor 162 is rotated around the central axis C, the support portion 246, the antibreak 244, and the insertion portion 242 of the endoscope 108 rotate around the central axis C. The second holder 124 and the second rotor 162 form the second operation element 118. The second operation element 118 can move the insertion portion 242 along its axial direction relative to the guide pipe 104 and the sheath 106, and can also rotate the insertion portion 242 around its axis. That is, the second operation element 118 can move the insertion portion 242 relative to the guide pipe 104 and the sheath 106.

Although the second holder 124 has the first rotor 162 in the example described here, the mechanism which rotates the insertion portion 242 of the endoscope 108 is not always needed.

The guide pipe 104 permits the distal side of the distal end 252a of the insertion portion 242 and the distal side of the distal end 104a to be observed by the observation optical system 264 of the endoscope 108 through the distal end 104a of the guide pipe 104. The guide pipe 104 permits the insertion portion 242 to be inserted therethrough so that the distal side of the distal end 252a of the insertion portion 242 can be observed by the observation optical system 264 while the distal end 252a of the insertion portion 242 of the endoscope 108 is protruding from the distal end 104a of the guide pipe 104.

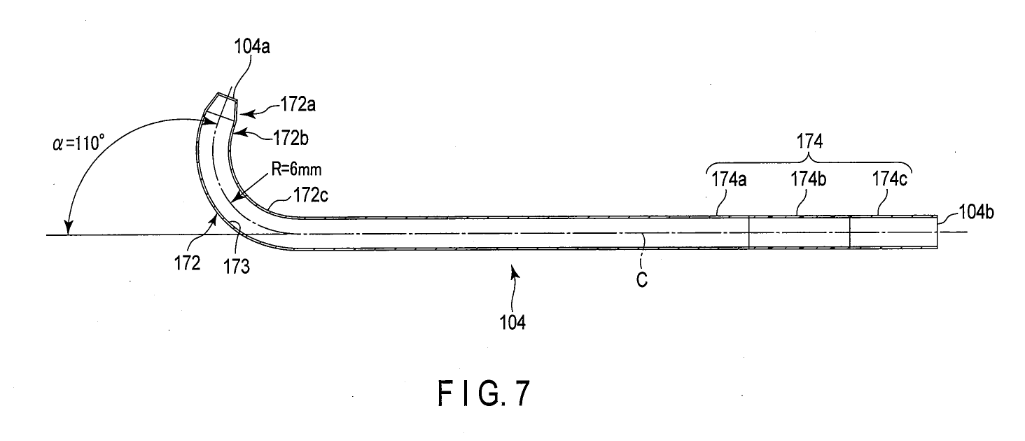

As shown in FIG. 7, the guide pipe 104 is formed by the bent pipe 172 and the straight pipe (guide pipe main body) 174 that are continuous with each other. The guide pipe 104 has an inside diameter (e.g., about 1.6 mm to 3.0 mm) which allows the insertion portion 242 of the endoscope 108 and the sheath body 152 of the sheath 106 to be inserted therethrough. The straight pipe (guide pipe main body) 174 is straight in the explanation here, but may naturally have a suitably bent part. The length of the straight pipe 174 is determined by the distance from an external nostril E to the nasal sinus to be observed/treated shown in FIG. 9B. For example, the distance from the external nostril E to an entrance G of the maxillary sinus M is about 30 mm to 90 mm.

The bent pipe 172 includes a distal end portion 172a which is tapered and which is fitted to, for example, the entrance G of the maxillary sinus M of the nasal sinuses, a guide portion 172b which is formed continuously with the distal end portion 172a, and the bent portion (guide pipe bent portion) 172c which is formed continuously with the guide portion 172b and which is integrated with the distal end of the straight pipe 174. It is preferable that, depending on the shape of the bent pipe 172, no guide portion 172b exists, and the distal end portion 172a may be formed continuously with the bent portion 172c. The bent portion 172c of the bent pipe 172 is bent at an angle α of, for example, about 110° to the straight pipe 174. As shown in FIG. 7, the guide pipe 104 in which the bent pipe 172 is bent is used for the observation or treatment of, for example, the maxillary sinus M of the nasal sinuses.

Here, a bending radius R of the bent pipe 172 of the guide pipe 104 shown in FIG. 7 is, for example, about 5 mm to 20 mm, preferably about 6 mm. The bending radius R of the bent portion 172c of the bent pipe 172 is set in consideration of the length (rigid length) toward the proximal side along the central axis C from the distal end face 252a of the distal hard portion 252 of the insertion portion 242 of the endoscope 108 in which the sheath body 152 of the sheath 106 is disposed on the outer circumference. Here, the guide pipe 104 has a larger inside diameter for observation by the endoscope 108 than when a simple guide wire or light guide fiber is used. In particular, the inside diameter of the straight pipe 174 can be smaller than the inside diameter of the bent pipe 172, but has substantially the same inside diameter to increase a suction performance.

The distal end portion 172a of the bent pipe 172 of the guide pipe 104 is tapered, so that the distal end (guide pipe distal opening) 104a of the guide pipe 104 is easily disposed from the near side to the far side of, for example, the entrance (opening) G (see FIG. 9A and FIG. 9B) of the maxillary sinus M of the nasal sinuses, in accordance with the size of, for example, the entrance G of the maxillary sinus M of the nasal sinuses. The distal end 104a of the guide pipe 104 is formed to have an inside diameter slightly larger than the outside diameter of the distal end 242a of the insertion portion 242 of the endoscope 108 so that the distal end 242a of the insertion portion 242 of the endoscope 108 can be put through the distal opening 104a. The inside diameter of the distal end 104a of the guide pipe 104 is preferably formed to be, for example, about 2 mm to 4 mm. The height (distance) of the distal end 104a of the guide pipe 104 relative to the central axis C of the straight pipe 174 of the guide pipe 104 is preferably about 20 mm or less. Various numerical values (e.g., the inside diameter and height) of the guide pipe 104 are suitably set in consideration of whether the guide pipe 104 can be inserted into a nasal cavity and in consideration of operability in the handling of the guide pipe 104.

The sheath 106 through which the insertion portion 242 of the endoscope 108 is inserted is movable relative to the straight pipe 174 of the guide pipe 104. The bent portion 172c of the bent pipe 172 of the guide pipe 104 is continuous with the distal side of the straight pipe 174, and has such an inside diameter and a bending radius that the distal hard portion 252 of the insertion portion 242 of the endoscope 108 can protrude to the distal side through the distal end 104a of the guide pipe 104 while the insertion portion 242 of the endoscope 108 is inserted through the sheath 106.

The straight pipe 174 of the guide pipe 104 may be made of a rigid material such as a metal (e.g., a stainless steel material or an aluminum alloy material) or a hard resin (e.g., polyethylene (PE) or polypropylene (PP)), or may be made of a combination of a rigid material and a flexible material such as silicone that are connected in the axial direction. In the latter case, the straight pipe 174 has a rigid portion 174a, an elastically deformable elastic portion 174b, and a proximal side rigid portion 174c. That is, the straight pipe 174 is at least partly elastically deformable. In the explanation here, the part of the straight pipe 174 from the proximal end of the rigid portion 174a (the distal end of the elastic portion 174b) to the distal end of the bent pipe 172 is seamlessly and integrally made of a stainless steel material. The elastic portion 174b may be formed at any position of the straight pipe 174 between the distal end and the proximal end. Due to this elastic portion 174b, the straight pipe 174 is elastically deformed in the elastic portion 174b if, for example, the distal end portion 172a of the bent pipe 172 or the distal opening 104a abuts on a living tissue. This can prevent the guide pipe 104 from applying a load to the living tissue.

The straight pipe 174 can provide similar advantageous effects even if the straight pipe 174 is formed to have, for example, suitable (given) flexibility. However, the straight pipe 174 of the guide pipe 104 is preferably higher in rigidity than the sheath 106 and the insertion portion 242 of the endoscope 108. Thus, the sheath 106 and the insertion portion 242 of the endoscope 108 that are inserted through the guide pipe 104 can be guided. In the guide pipe 104, not only the straight pipe 174 but also the bent pipe 172 is preferably made of a resinous material which is higher in rigidity than the sheath 106 and the insertion portion 242 of the endoscope 108 and which has suitable flexibility.

As shown in FIG. 5A, an adapter 175 is fixed to a proximal end (proximal side opening) 104b of the elastic portion 174b of the straight pipe 174 of the guide pipe 104. The adapter 175 is fixed to the connecting pipe 120 by a fixing body 175a such as a screw. An O-ring 121a is provided between the connecting pipe 120 and the adapter 175 of the guide pipe 104. Thus, the space between the guide pipe 104 and the adapter 175 is sealed. Therefore, the space between the inner circumferential surface of the guide pipe 104 and the adapter 175 can be airtight and/or liquid-tight.

The distal end 104a of the guide pipe 104 is preferably formed into a rounded shape. This prevents a load from being put on the mucous membrane inside the nose.

A balloon (not shown) which can be elastically deformed into, for example, a ring-shape is preferably disposed on the outer circumferential surface of the distal end portion 172a of the bent pipe 172. If the balloon is inflated, the outer circumferential surface of the distal end portion 172a of the guide pipe 104 can be held around the opening of, for example, the maxillary sinus M of the nasal sinuses relative to the entrance of the nasal sinuses. Thus, by the inflation of the balloon, it is possible to prevent the distal end portion 172a of the guide pipe 104 from being displaced relative to, for example, the entrance G of the maxillary sinus M of the nasal sinuses.

It is also preferable to use a flexible ring (not shown) made of, for example, a rubber material instead of the balloon. As in the case of the use of the balloon, the ring can be kept elastically deformed, for example, in the slightly large entrance G of the maxillary sinus M of the nasal sinuses, and can prevent the distal end 104a of the guide pipe 104 from being pulled out of the entrance of the nasal sinus, and prevent the guide pipe 104 from excessively entering the nasal sinus, as the position of the distal end 252a of the insertion portion 242 of the endoscope 108 may otherwise be unstable.

Here, for simplicity of explanation, the bending direction of the bent pipe 172 relative to the straight pipe 174 of the guide pipe 104 is defined to be upward (see FIG. 1B) relative to the main body 112 of the handle unit 102.

In this embodiment, the bending direction of the bent pipe 172 relative to the straight pipe 174 of the guide pipe 104 is upward (the opening direction of the main body 112 of the handle unit 102), but such can be set according to user preference.

As shown in FIG. 1A to FIG. 2B, the first and second operation elements 116 and 118 are linked by an interlock portion (synchronizing portion) 200 so that their operations are synchronized. The interlock portion 200 may be made of a resinous material or a metallic material. The interlock portion 200 is preferably used when the sheath 106 and the endoscope 108 are set to the guide pipe 104. As required, the interlock portion 200 is preferably used when the inside of the later-described maxillary sinus M is observed and treated by the guide pipe 104.

The interlock portion 200 includes a first support portion 202 which supports the first operation element 116, a second support portion 204 which supports the second operation element 118, and a linking body 206 which links the first and second support portions 202 and 204. Thus, the interlock portion 200 keeps the first and second operation elements 116 and 118 apart from each other by a predetermined distance. The interlock portion 200 then keeps the distal end 104a of the guide pipe 104 and the distal end 106a of the sheath 106 substantially in place. It is also preferable that the interlock portion 200 can adjust the positions of the first and second support portions 202 and 204 relative to the linking body 206. In this case, the interlock portion 200 can set a suitable state even if the sheath 106 and the insertion portion 242 of the endoscope 108 change in length due to, for example, air temperature.

If the interlock portion 200 is moved along the axial direction of the central axis C, the first operation element 116 and the second operation element 118 move in the same direction together. Thus, if the interlock portion 200 is moved forward relative to the handle unit 102 along the central axis C, the first and second operation elements 116 and 118 move forward together. If the interlock portion 200 is moved backward relative to the handle unit 102 along the central axis C, the first and second operation elements 116 and 118 move backward together. This is the same even if the first operation element 116 or the second operation element 118 is operated instead of the interlock portion 200.

The interlock portion 200 can be easily detached from the first and second operation elements 116 and 118. In particular, the interlock portion 200 can be detached with one hand when the user is holding the handle unit 102. It is also easy to again attach the interlock portion 200 by adjusting the distance between the first and second operation elements 116 and 118.

The interlock portion 200 is preferably connected to the handle unit 102 by an unshown string. In this case, the time for searching for the detached interlock portion 200 can be saved.

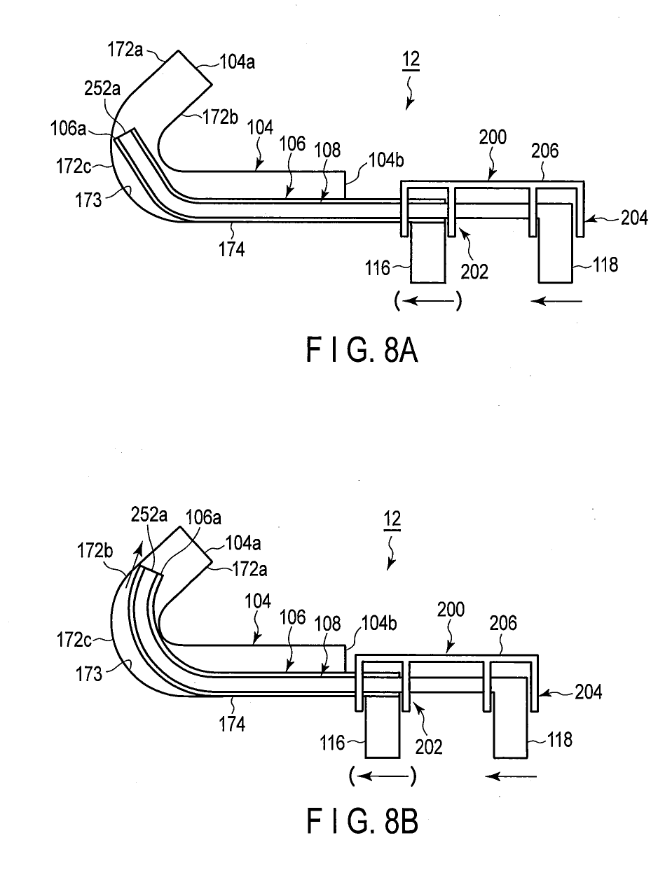

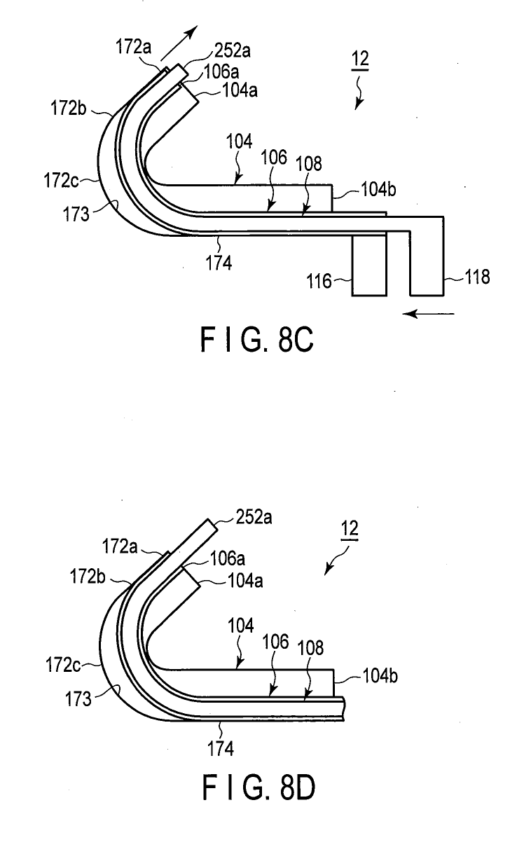

Now, the functions of the treatment system 10 according to this embodiment are described with reference to FIG. 8A to FIG. 9B.

Schematic diagrams of the interlock portion 200 for the guide pipe 104, the sheath 106, and the endoscope 108 are shown in FIG. 8A and FIG. 8B. Although described in detail later, FIG. 8A shows the situation in which the distal end 106a of the sheath 106 and the distal end 252a of the insertion portion 242 of the endoscope 108 are located in the bent portion 172c of the bent pipe 172 of the guide pipe 104 when the interlock portion 200 is operated to move the sheath 106 and the insertion portion 242 of the endoscope 108 forward. FIG. 8B shows the situation immediately after the distal end 106a of the sheath 106 and the distal end 252a of the insertion portion 242 of the endoscope 108 have gone beyond the bent portion 172c of the bent pipe 172 of the guide pipe 104. FIG. 8C shows the situation in which the insertion portion 242 of the endoscope 108 is moved forward while the interlock portion 200 is detached from the handle unit 102 in the situation shown in FIG. 8B and then the position of the sheath 106 is held relative to the guide pipe 104. FIG. 8D shows the situation in which the insertion portion 242 of the endoscope 108 is further moved forward from the situation shown in FIG. 8C so that the distal end 252a of the insertion portion 242 of the endoscope 108 protrudes from the distal end 104a of the guide pipe 104 and the distal end 106a of the sheath 106. As shown in FIG. 8C and FIG. 8D, when the distal end 106a of the sheath 106 and the distal end 252a of the insertion portion 242 of the endoscope 108 are located beyond the bent portion 172c of the bent pipe 172 of the guide pipe 104, the distal end 106a of the sheath 106 and the distal end 252a of the insertion portion 242 of the endoscope 108 can be freely moved while being kept located beyond the bent portion 172c of the bent pipe 172 of the guide pipe 104.

In the example described here, the treatment system 10 is used to observe and treat, for example, the maxillary sinus M of the nasal sinuses. That is, a series of treatments using the treatment system 10 are conducted as follows:

(Step 1) The insertion device assembly 12 of the treatment system 10 is prepared as below.

As shown in FIG. 1A to FIG. 2B, the insertion portion 242 of the endoscope 108 is inserted through the guide pipe 104 and the sheath 106 to form the insertion device assembly 12. As shown in FIG. 1A, the cable 248 of the endoscope 108 of the insertion device assembly 12 is connected to the controller 14. In addition, the joint 136 of the handle unit 102 is connected to the change-over valve 28, and the treatment system 10 is formed.

As shown in FIG. 5A, the friction between the outer circumferential surface of the sheath 106 and the O-ring 120a disposed in the connecting pipe 120 and the friction between the O-ring 138a disposed in the first operation element 116 and the first rotor 134 inhibit the first operation element 116 from unintentionally rotating due to, for example, gravity and unintentionally moving along the central axis C. Similarly, as shown in FIG. 5B, the friction between the outer circumferential surface of the insertion portion 242 of the endoscope 108 and the O-ring 138b disposed in the first operation element 116 inhibits the second operation element 118 from unintentionally rotating due to, for example, gravity and unintentionally moving along the central axis C. In this instance, a frictional force between the inner circumferential surface of the O-ring 138b and the outer circumferential surface of the insertion portion 242 of the endoscope 108 is set to such a degree that the first operation element 116 and the second operation element 118 independently operate.

When the interlock portion 200 is not fitted into the first operation element 116 and the second operation element 118, the interlock portion 200 is suitably fitted as shown in FIG. 1A to FIG. 2B. The length of the sheath 106 and the length of the insertion portion 242 of the endoscope 108 are suitably adjusted, so that the distal end 106a of the sheath 106 and the distal end 252a of the insertion portion 242 of the endoscope 108 are substantially in place, that is, correspond or come closer as shown in FIG. 8A and FIG. 8B if the interlock portion 200 is suitably fitted to the first operation element 116 and the second operation element 118. In this instance, the distal end 252a of the insertion portion 242 of the endoscope 108 may slightly protrude from or may be slightly retracted relative to the distal end 106a of the sheath 106. The interlock portion 200 sets the distance between the first operation element 116 and the second operation element 118 so that the endoscope 108 can observe the outside through the distal opening 106a of the sheath 106 by the observation optical system 264 of the insertion portion 242 even if the distal end 252a of the insertion portion 242 of the endoscope 108 is slightly retracted relative to the distal end 106a of the sheath 106. The interlock portion 200, the first operation element 116, and the second operation element 118 operate in dependence on one another. That is, the sheath 106 and the endoscope 108 are moved relative to the guide pipe 104 and the handle unit 102 while the distal end 106a of the sheath 106 and the distal end 252a of the insertion portion 242 of the endoscope 108 are substantially in place.

A state in which the first operation element 116 and the second operation element 118 are located on the most proximal side of the handle unit 102 while the interlock portion 200 is disposed in the first operation element 116 and the second operation element 118 is a neutral state. In this instance, the distal end 106a of the sheath 106 and the distal end 252a of the insertion portion 242 of the endoscope 108 are located inside the straight pipe (guide pipe main body) 174 of the guide pipe 104. A proximal side surface 173 of the bent portion 172c of the bent pipe 172 of the guide pipe 104 can be observed by the endoscope 108, but the guide portion 172b and the distal end portion 172a cannot be observed.

As shown in FIG. 8A, the user moves the interlock portion 200 relative to the guide pipe 104 from the neutral state so that the distal end 106a of the sheath 106 and the distal end 252a of the insertion portion 242 of the endoscope 108 pass through the bent portion 172c of the bent pipe 172 of the guide pipe 104. In this instance, the distal end 106a of the sheath 106 and the distal end 252a of the insertion portion 242 of the endoscope 108 abut on the proximal side surface 173 of the bent portion 172c of the bent pipe 172 of the guide pipe 104.

The sheath 106 has an adequate flexibility and an adequate difficulty of breaking. Thus, if the sheath 106 is moved forward along the guide pipe 104 and then abuts on the proximal side surface 173 of the bent portion 172c of the bent pipe 172, the distal end 106a of the sheath 106 is directed toward the guide portion 172b in response to a reaction force from the proximal side surface 173. In this instance, the part (distal end 152a) located in the vicinity of the distal end 106a of the sheath 106 is substantially straight because the distal hard portion 252 exists at the distal end 252a of the insertion portion 242 of the endoscope 108.

As shown in FIG. 8B, if the interlock portion 200 is further moved forward with respect to the guide pipe 104, the distal end 106a of the sheath 106 and the distal end 252a of the insertion portion 242 of the endoscope 108 are bent in the same direction as the bending direction of the bent pipe 172, that is, toward the guide portion 172b along the proximal side surface 173 of the bent pipe 172 of the guide pipe 104. The distal hard portion 252 of the endoscope 108 then passes through the bent portion 172c of the bent pipe 172 together with the distal end 106a of the sheath 106. If the distal opening 104a of the guide pipe 104 is observed by the endoscope 108, it is possible to recognize that the distal end 106a of the sheath 106 and the distal end 252a of the insertion portion 242 of the endoscope 108 have passed through the bent portion 172c of the bent pipe 172. In this instance, the distal end 106a of the sheath 106 and the distal end 252a of the insertion portion 242 of the endoscope 108 have passed through the bent portion 172c of the guide pipe 104 and are located between the bent portion 172c and the distal opening 104a, that is, located in the guide portion 172b or the distal end portion 172a.

When the distal end 106a of the sheath 106 and the distal hard portion 252 of the endoscope 108 do not pass through the bent portion 172c of the bent pipe 172 of the guide pipe 104, the first and second rotors 134 and 162 (see FIG. 2A, FIG. 2B, FIG. 4A, and FIG. 4B) of the handle unit 102 are suitably moved to suitably rotate the sheath 106 and the insertion portion 242 of the endoscope 108. As described above, the distal hard portion 252 of the endoscope 108 is put through the bent portion 172c of the bent pipe 172 together with the distal end 106a of the sheath 106.

As shown in FIG. 8C, if the distal hard portion 252 of the insertion portion 242 of the endoscope 108 has passed through the bent portion 172c of the bent pipe 172, the interlock portion 200 is detached from the first operation element 116 and the second operation element 118. In this instance, the O-ring 120a shown in FIG. 5A prevents the sheath 106 from unintentionally moving relative to the handle unit 102 and the guide pipe 104, and the O-ring 138b shown in FIG. 5B prevents the insertion portion 242 of the endoscope 108 from unintentionally moving relative to the handle unit 102 and the guide pipe 104.

As shown in FIG. 8D, the second operation element 118 is then operated so that the distal end 252a of the insertion portion 242 of the endoscope 108 protrudes from the distal end 106a of the sheath 106 and the distal end 104a of the guide pipe 104. When the distal end 252a of the insertion portion 242 of the endoscope 108 protrudes out of the guide pipe 104, the situation outside the guide pipe 104 can be suitably observed by the observation optical system 264 of the insertion portion 242 of the endoscope 108. In this case, because the blockage of the field of view for the distal end 104a of the guide pipe 104 and the distal end 106a of the sheath 106 is prevented, the observation optical system 264 of the insertion portion 242 of the endoscope 108 can observe over a wider range. That is, by suitably moving the first and second operation elements 116 and 118, the user can suitably observe the situation outside the guide pipe 104 while observing the distal end 104a of the guide pipe 104 and the distal end 106a of the sheath 106 using the observation optical system 264 of the insertion portion 242 of the endoscope 108. In this instance, if it is difficult to locate the insertion portion 242 of the endoscope 108 at a desired position due to its flexibility, the first operation element 116 is operated to bring the distal end 106a of the sheath 106 closer to the distal end 252a of the insertion portion 242 of the endoscope 108. In this instance, the distal end 252a of the insertion portion 242 of the endoscope 108 can be kept in a suitable state by the flexibility and the difficulty of breaking of the sheath 106.

The distal end 252a of the insertion portion 242 of the endoscope 108 is then slightly drawn into the distal end 104a of the guide pipe 104, further into the state shown in FIG. 8C. In this instance, the endoscope 108 can observe the outside through the distal opening 104a of the guide pipe 104.

As shown in FIG. 9A and FIG. 9B, in the situation in which the position of the distal end 106a of the sheath 106 and the position of the distal end 252a of the insertion portion 242 of the endoscope 108 are thus adjusted relative to the bent pipe 172 of the guide pipe 104, the user then inserts the distal end 104a of the guide pipe 104 of the insertion device assembly 12 into the nasal sinus through the external nostril E of the nose N. This operation will be described later.

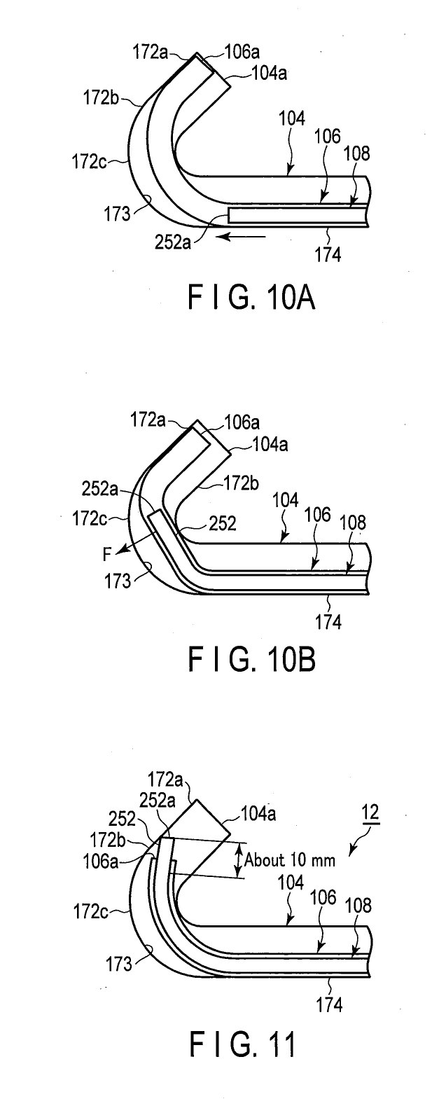

Now, an example of how the sheath 106 and the endoscope 108 are used for the guide pipe 104 without the interlock portion 200 is described with reference to FIG. 10A and FIG. 10B. In this case, it is difficult for the user to simultaneously move the first operation element 116 and the second operation element 118 together with the handle unit 102. Thus, the user independently moves the first operation element 116 and the second operation element 118.

First, the first and second operation elements 116 and 118 are operated to move the distal end 106a of the sheath 106 and the distal end 252a of the insertion portion 242 of the endoscope 108 to the most proximal side relative to the handle unit 102. For example, as shown in FIG. 10A, the sheath 106 precedes the insertion portion 242 of the endoscope 108 so that the distal end 106a of the sheath 106 is brought closer to the distal opening 104a of the guide pipe 104. In this instance, the distal end 106a of the sheath 106 has passed through the bent portion 172c of the bent pipe 172 and is located in the distal end portion 172a or the guide portion 172b, and the distal hard portion 252 of the insertion portion 242 of the endoscope 108 has not passed through the bent portion 172c of the bent pipe 172 of the guide pipe 104.

The sheath 106 is formed to be more firm and more unbendable than the flexible pipe 254 of the insertion portion 242 of the endoscope 108. Thus, when the distal end 106a of the sheath 106 has passed through the bent portion 172c, resilience F works such that the part of the sheath 106 which has passed through the bent portion 172c becomes straight again. In this state, the second operation element 118 is operated relative to the handle unit 102 so that the distal hard portion 252 of the insertion portion 242 of the endoscope 108 will pass through the bent portion 172c of the bent pipe 172 of the guide pipe 104 as shown in FIG. 10B. In this instance, force F that brings the flexible pipe 254 closer to the straight state is applied to the distal hard portion 252 of the endoscope 108 in the sheath 106 by the resilience F of the sheath 106.

Thus, as shown in FIG. 10B, when the distal hard portion 252 of the insertion portion 242 of the endoscope 108 is inserted toward the distal end 106a of the sheath 106, the sheath 106 applies the force F that presses the distal hard portion 252 of the insertion portion 242 of the endoscope 108 toward the proximal side surface 173 of the bent portion 172c. Therefore, it is difficult or impossible to cause the distal hard portion 252 of the insertion portion 242 of the endoscope 108 to pass through the bent portion 172c of the bent pipe 172 of the guide pipe 104. In any case, a load is applied to the insertion portion 242 of the endoscope 108, particularly to the part located in the vicinity of the boundary between the distal hard portion 252 and the flexible pipe 254 by the sheath 106.

The flexible pipe 254 of the insertion portion 242 of the endoscope 108 is not firm in contrast to the sheath 106. Thus, when the distal end 252a of the insertion portion 242 of the endoscope 108 is brought closer to the distal opening 104a through the bent pipe 172 of the guide pipe 104 without the use of the sheath 106, the flexible pipe 254 of the insertion portion 242 of the endoscope 108 might be corrugated inside the straight pipe (guide pipe main body) 174 of the guide pipe 104. When the distal end 252a of the insertion portion 242 of the endoscope 108 passes through the bent portion 172c of the bent pipe 172 of the guide pipe 104 without the use of the sheath 106, the distal hard portion 252 does not face toward the guide portion 172b, and a load might be applied to the distal hard portion 252 of the endoscope 108. Thus, when the distal end 252a of the insertion portion 242 of the endoscope 108 passes through the bent portion 172c of the bent pipe 172 of the guide pipe 104, the distal end 252a of the insertion portion 242 of the endoscope 108 is preferably covered with the sheath 106.

(Step 2) The distal end 104a of the guide pipe 104 and the distal end 106a of the sheath 106 of the insertion device assembly 12 of the treatment system 10, and the distal end 252a of the insertion portion 242 of the endoscope 108 are inserted into the entrance (opening) G of the nasal sinus to be treated, particularly, the maxillary sinus M shown in FIG. 9A and FIG. 9B from the external nostril E.

The user guides the distal end 104a of the guide pipe 104 of the insertion device assembly 12, for example, to the entrance G of the maxillary sinus M of the nasal sinuses from the external nostril E while maintaining the state (the state shown in FIG. 8C in particular) described above in (step 1). In this instance, the part which the distal end 104a of the guide pipe 104 is facing is displayed on the monitor 16. The user visually checks the monitor 16 while operating the handle unit 102 and thus suitably moving the distal end 104a of the guide pipe 104, and thereby observes an insertion path from the external nostril E to the entrance G of the maxillary sinus M in detail. In this instance, the application of a large force to the insertion path is prevented by the elastic deformation of the elastic portion 174b of the straight pipe 174 even if the insertion path from the external nostril E to the maxillary sinus M is unintentionally pressed by the bent pipe 172 of the guide pipe 104.

The entrance G of the maxillary sinus M is located in the middle meatus, and is open in the back surface of the coronoid process. The entrance G of the maxillary sinus M may be blocked by the coronoid process. The user suitably moves the handle unit 102, and turns the distal opening 104a of the bent pipe 172 of the guide pipe 104 having a bending angle of 110°, and then recognizes the coronoid process on the monitor 16 by the endoscope 108. In this way, the user finds the entrance G of the maxillary sinus M in the back surface of the coronoid process by the endoscope 108. The distal end 104a of the guide pipe 104 is then moved and inserted into the entrance G of the maxillary sinus M which has been found by the endoscope 108.

The distal end portion 172a including the distal end 104a of the guide pipe 104 is thus inserted and located in the entrance G of the maxillary sinus M of the nasal sinuses so that the distal end 252a of the insertion portion 242 of the endoscope 108 and/or the distal end 106a of the sheath 106 can access the inside of the maxillary sinus M. The user observes and treats the inside of the maxillary sinus M while keeping the distal end portion 172a of the guide pipe 104 at the entrance G of the maxillary sinus M as much as possible.

The distal end 252a of the insertion portion 242 of the endoscope 108 may be displaced relative to the distal end 104a of the guide pipe 104 while the distal end 104a of the guide pipe 104 is guided to the entrance G of the maxillary sinus M of the nasal sinuses from the external nostril E. In this case, as has been described above in (step 1), the distal end 252a of the insertion portion 242 of the endoscope 108 is disposed in the vicinity of the distal end 104a of the guide pipe 104 by the use of the interlock portion 200. Thus, the interlock portion 200 may also be attached to the handle unit 102 while the distal end 104a of the guide pipe 104 is guided to the entrance G of the maxillary sinus M of the nasal sinuses from the external nostril E.

When the distal end 252a of the insertion portion 242 of the endoscope 108 has come to a dead end while the distal end portion 172a of the guide pipe 104 is kept at the entrance G of the maxillary sinus M, an observation image showing the color of a mucous membrane is displayed on the entire monitor 16. On the other hand, when there is an insertion path ahead of the distal end 252a of the insertion portion 242 of the endoscope 108, not only the mucous membrane but also a passage through a narrowed area is displayed on the monitor 16.

A case in which the insertion path from the entrance G of the maxillary sinus M into the maxillary sinus M is narrow is briefly described as a first condition.

The interlock portion 200 is detached, and the second operation element 118 is then brought closer to the first operation element 116. The distal end 252a of the insertion portion 242 of the endoscope 108 is protruded from the distal end 104a of the guide pipe 104 and the distal end 106a of the sheath 106. In this instance, the distal end 252a of the insertion portion 242 is put through the narrow path. After or as the distal end 252a of the insertion portion 242 passes through the narrow path, the first operation element 116 is moved toward the distal end 112a of the main body 112 of the handle unit 102, and the distal end 106a of the sheath 106 is brought closer to the distal end 252a of the insertion portion 242. In this instance, the distal end 106a of the sheath 106 and the distal end 252a of the insertion portion 242 of the endoscope 108 are located inside the maxillary sinus M.

A case in which the insertion path from the entrance G of the maxillary sinus M into the maxillary sinus M is big enough for both the distal end 252a of the insertion portion 242 and the distal end 106a of the sheath 106 to pass through is briefly described as a second condition.

While the first and second operation elements 116 and 118 are interlocked by the interlock portion 200, the interlock portion 200 and the first operation element 116 or the second operation element 118 are moved toward the distal end 112a of the main body 112 of the handle unit 102. In this instance, the distal end 106a of the sheath 106 and the distal end 252a of the insertion portion 242 of the endoscope 108 are located inside the maxillary sinus M. The interlock portion 200 is then detached from the first and second operation elements 116 and 118.

A case in which viscous matter adheres to the insertion path from the entrance G of the maxillary sinus M into the maxillary sinus M is briefly described as a third condition.

When the user is recognized in the observation image by the endoscope 108 that viscous matter is adhering to the insertion path, the first operation element 116 is moved forward relative to the handle unit 102, and the distal end 106a of the sheath 106 is moved to a position located in the vicinity of the viscous matter simultaneously with the observation of the observation image. If necessary, the second operation element 118 is moved forward relative to the handle unit 102 so that the viscous matter can be observed by the endoscope 108. In this instance, the user can observe the distal side of the distal end 252a of the insertion portion 242 by the observation optical system 264 through the distal end 106a of the sheath 106.

After the end of the observation, the second operation element 118 is retreated relative to the handle unit 102 while the position of the first operation element 116 is maintained. That is, while the distal end 106a of the sheath 106 is protruding from the distal end 104a of the guide pipe 104, the distal end 252a of the insertion portion 242 of the endoscope 108 is retracted relative to the distal end 106a of the sheath 106. In this instance, the distal end 252a of the insertion portion 242 of the endoscope 108 is located between the bent portion 172c of the guide pipe 104 and the distal end 104a. The suction source 22 shown in FIG. 1A is then operated to suck the viscous matter from the distal end 106a of the sheath 106 through the space between the inner circumferential surface of the sheath 106 and the outer circumferential surface of the insertion portion 242 of the endoscope 108.

When the suction of the viscous matter is difficult, the second operation element 118 may be retreated to the proximal side relative to the handle unit 102, the inner bore of the sheath 106 may be released, and suction may be performed. In this instance, the second operation element 118 is retreated to the most proximal side relative to the handle unit 102. The distal end 252a of the insertion portion 242 of the endoscope 108 is then located slightly closer to the proximal side than the position of the joint 136 of the handle unit 102. The suction source 22 shown in FIG. 1A is then operated to suck the viscous matter from the distal end 106a of the sheath 106 through the inner circumferential surface of the sheath 106. In this case, larger-size matter can be sucked because the insertion portion 242 of the endoscope 108 is not present in the suction path.

After the operation of the suction source 22 is stopped, the first and second operation elements 116 and 118 are put in the neutral state as has been described above in (step 1). The interlock portion 200 is then used to locate the distal end 106a of the sheath 106 and the distal end 252a of the insertion portion 242 of the endoscope 108 in the vicinity of the distal end 104a of the guide pipe 104. The distal end 106a of the sheath 106 and the distal end 252a of the insertion portion 242 of the endoscope 108 are then located inside the maxillary sinus M.

(Step 3) The observation image by the endoscope 108 is then used to diagnose the inside of the maxillary sinus M of the nasal sinuses. Although the distal end portion 172a including the distal opening 104a of the guide pipe 104 is kept inserted in the entrance G of the maxillary sinus M in the case described here for simplicity of explanation, the distal end portion 172a of the guide pipe 104 may be located away from the entrance G of the maxillary sinus M as shown in FIG. 9B. In this case, it is preferable that at least one of the distal end 106a of the sheath 106 and the distal end 252a of the insertion portion 242 of the endoscope 108 is located inside the maxillary sinus M.

As has been described above in (step 2), the distal end 106a of the sheath 106 and the distal end 252a of the insertion portion 242 of the endoscope 108 are located inside the maxillary sinus M. The second operation element 118 is moved forward relative to the handle unit 102 so that the distal end 252a of the insertion portion 242 of the endoscope 108 protrudes from the distal end 106a of the sheath 106. In this state, while the second operation element 118 is being moved to move the distal end 252a of the insertion portion 242 of the endoscope 108, the monitor 16 is observed to check the state inside the maxillary sinus M; for example, whether viscous matter is retained, the state of the surface of the mucous membrane, the color and state of mucus, and the state of the mucous membrane. The user suitably rotates the second rotor 162 of the second operation element 118 around the central axis C to rotate a figure displayed on the monitor 16 in a suitable direction if necessary.

When viscous matter exists inside the maxillary sinus M, the user checks its color and volume on the monitor 16. When the viscous matter is sucked and removed, the user operates the suction source 22 to perform suction as described above while keeping the distal end 106a of the sheath 106 inside the maxillary sinus M.

After the suction source 22 is stopped, the second operation element 118 is brought closer to the second operation elements 116 to insert the distal end 252a of the insertion portion 242 of the endoscope 108 into the nasal sinus beyond the distal end 106a of the sheath 106. In this instance, if the distal end 252a of the insertion portion 242 of the endoscope 108 is evacuated to the rear of the joint 136, the interlock portion 200 is used to locate the distal end 106a of the sheath 106 and the distal end 252a of the insertion portion 242 of the endoscope 108 in the vicinity of the distal end 104a of the guide pipe 104 while the first and second operation elements 116 and 118 are put in the neutral state, as has been described above in (step 1). The first and second operation elements 116 and 118 are then operated to bring the distal end 106a of the sheath 106 and the distal end 252a of the insertion portion 242 of the endoscope 108 into the maxillary sinus M. The colors and states of the mucous membrane and the viscous matter inside the nasal sinus and the volume of the viscous matter are then checked again on the monitor 16. In this instance, the first and second operation elements 116 and 118 are suitably moved back and forth, and the second rotor 162 of the second operation element 118 is suitably rotated to check the inside of the maxillary sinus M. In particular, inflamed parts of the mucous membrane inside the maxillary sinus M are checked.

In this way, the user observes the inside of the maxillary sinus M by the use of the endoscope 108 to diagnose the state of the affected part.

(Step 4) The inside of the nasal sinuses is cleaned as required.

A liquid such as a physiological saline is put into the maxillary sinus M from the liquid supply source 24. If a greater volume of the supplied liquid is needed, the distal end 252a of the insertion portion 242 is pulled out of the guide pipe 104 through the distal end 106a of the sheath 106 and the distal end 104a of the guide pipe 104 while the distal end 106a of the sheath 106 is maintained in the nasal sinus. The distal end 252a of the insertion portion 242 of the endoscope 108 is then evacuated to the rear of the joint 136.

When the liquid is supplied from the liquid supply source 24 in this condition, a greater volume of liquid can be supplied than when the insertion portion 242 of the endoscope 108 is located inside the sheath 106. Thus, a wider range can be cleaned if this insertion device assembly 12 is used.

After the inside of the maxillary sinus M is cleaned with the physiological saline (cleaning liquid) in this way, the physiological saline containing the viscous matter is then sucked by the suction source 22.

As has been described above in (step 1), the interlock portion 200 is then used to locate the distal end 106a of the sheath 106 and the distal end 252a of the insertion portion 242 of the endoscope 108 in the vicinity of the distal end 104a of the guide pipe 104 while the first and second operation elements 116 and 118 are put in the neutral state. The first and second operation elements 116 and 118 are then operated to bring the distal end 106a of the sheath 106 into the maxillary sinus M and bring the distal end 252a of the insertion portion 242 of the endoscope 108 into the maxillary sinus M. The second rotor 162 of the second operation element 118 is then suitably rotated to check the color and volume of the mucous membrane inside the maxillary sinus M on the monitor 16.

If there is any viscous matter remaining in the maxillary sinus M, the viscous matter is sucked. When there is any viscous matter that cannot be sucked or when a particular mucous membrane tissue needs to be extracted, the endoscope 108 is detached from the handle unit 102 while the distal end 106a of the sheath 106 is maintained in the maxillary sinus M. The pin 164 disposed in the plane 246a of the support portion 246 of the endoscope 108 is then removed to detach the insertion portion 242 of the endoscope 108 from the insertion device assembly 12. The insertion portion 242 of the endoscope 108 is then pulled out of the sheath 106.

A forceps or a brush, for example, is put through the sheath 106 from the proximal side of the T-shaped pipe 132, and inserted into the maxillary sinus M beyond the distal end 106a of the sheath 106. In this condition, the mucous membrane tissue inside the maxillary sinus M may be extracted by, for example, the forceps or the brush. The forceps or the brush can also be used instead of suction to collect the viscous matter.

(Step 5) The inside of the nasal sinuses is treated. For example, medicine is administered to the affected part inside the maxillary sinus M.

The forceps or the brush, for example, is removed from the sheath 106, and the insertion portion 242 of the endoscope 108 is again attached to the handle unit 102. As has been described above in (step 1), the interlock portion 200 is used to locate the distal end 106a of the sheath 106 and the distal end 252a of the insertion portion 242 of the endoscope 108 in the vicinity of the distal end 104a of the guide pipe 104 while the first and second operation elements 116 and 118 are put in the neutral state. The sheath 106 and the insertion portion 242 of the endoscope 108 are suitably moved to check the affected part.

The chemical is stuck to the affected part from the liquid supply source 24 through the space between the inner circumferential surface of the sheath 106 and the outer circumferential surface of the insertion portion 242. In this instance, the chemical to be supplied is, for example, a steroid and/or an antibacterial agent. Moreover, the supplied chemical is preferably retained in the maxillary sinus M after the supply. To supply the chemical, for example, a medicine such as a steroid and/or an antibacterial agent is contained in temperature-responding gel which increases viscosity at about body temperature. Since the chemical increases viscosity after being supplied, the chemical can be in contact with the affected part for a long time. The chemical is then retained in the maxillary sinus M so that the effect of the chemical can be prolonged.

If necessary, the chemical is administered to the affected part while the insertion portion 242 of the endoscope 108 is pulled out of the sheath 106.

(Step 6) The insertion device assembly 12 of the endoscopic treatment system 10 is pulled out of the maxillary sinus M.

The first and second operation elements 116 and 118 are moved toward the proximal end 112b of the main body 112 of the handle unit 102. Thus, the distal end 252a of the insertion portion 242 of the endoscope 108 and the distal end 106a of the sheath 106 are pulled out of the maxillary sinus M. The distal end 106a of the sheath 106 and the distal end 252a of the insertion portion 242 of the endoscope 108 are then retracted relative to the distal end 104a of the guide pipe 104. When the balloon is inflated, the balloon is deflated.

The distal end portion 172a of the bent pipe 172 of the guide pipe 104 disposed at the entrance G of the maxillary sinus M is separated from the entrance G of the maxillary sinus M with the possible effort to prevent abutment with the mucous membrane tissue inside the nose, and the guide pipe 104 is then pulled out of the nostril.

After the end of a series of observations and treatments, the user removes the pin 164 disposed in the plane 246a of the support portion 246 of the endoscope 108, and then detaches the endoscope 108 from the handle unit 102. The endoscope 108 is then cleaned, disinfected, and sterilized to be reused. The guide pipe 104 may be disassembled and then cleaned, disinfected, and sterilized to be reused or may be simply disposed of. The sheath 106 is detached from the handle unit 102 and then disposed of.

As described above, the following can be said according to the treatment system 10 according to this embodiment.

The treatment system 10 according to this embodiment is used to treat the nasal sinuses through the nose of the patient. Because the guide pipe 104, the sheath 106, and the endoscope 108 of the insertion device assembly 12 for nasal sinuses need to access the entrance of the nasal sinus (e.g., the entrance G of the maxillary sinus M) from the external nostril E, the guide pipe 104, the sheath 106, and the endoscope 108 have portions that are extremely small in diameter. When the insertion portion 242 of the endoscope 108 is put through the straight pipe (main body) 174 and then put through the guide pipe 104 in which the bent portion 172c of the bent pipe 172 is at more than 90°, for example, 110°, a load might be applied to the insertion portion 242 of the endoscope 108 having an extremely small diameter. It is therefore necessary to put the sheath 106 and the insertion portion 242 of the endoscope 108 through the bent portion 172c of the bent pipe 172 while protecting the insertion portion 242 of the endoscope 108 with the sheath 106 and at the same time preventing the application of a load to the insertion portion 242 of the endoscope 108 by the sheath 106. In the insertion device assembly 12 according to this embodiment, the interlock portion 200 can be used to move the distal end 106a of the sheath 106 and the distal end 252a of the insertion portion 242 of the endoscope 108 so that their positions correspond to or come closer to each other. Thus, the interlock portion 200 can be used to protect the insertion portion 242 of the endoscope 108 with the sheath 106. The use of the interlock portion 200 can prevent the resilience of the sheath 106 from becoming a hindrance when the distal hard portion 252 of the insertion portion 242 of the endoscope 108 passes through the bent portion 172c of the bent pipe 172. That is, the use of the interlock portion 200 can prevent the application of a load to the insertion portion 242 of the endoscope 108 by the sheath 106. Since it is not necessary to independently insert the insertion portion 242 of the endoscope 108 through the guide pipe 104, the distal end 252a of the insertion portion 242 of the endoscope 108 can be protected with the sheath 106, and the application of a load to the insertion portion 242 of the endoscope 108 can be inhibited.

The insertion device assembly 12 is used to display the observation image on the monitor 16, and the user can dispose the distal end portion 172a of the guide pipe 104, for example, at the entrance G of the maxillary sinus M of the nasal sinuses with certainty while viewing the observation image. In this instance, the endoscope 108 is used, so that the user (doctor) can easily recognize the situation in the insertion path (e.g., the condition of the mucous membrane in the nose). Therefore, by using the insertion device assembly 12 according to this embodiment, it is possible for the distal end 104a of the guide pipe 104 to safely and surely access the entrance of the nasal sinus while the situation in the path from the external nostril E to the nasal sinuses is recognized by the observation image. Because the endoscope 108 is used, it is possible to safely and certainly recognize whether a desired nasal sinus such as the maxillary sinus M can be accessed. Therefore, by using the insertion device assembly 12 according to this embodiment, it is possible for the distal end 104a of the guide pipe 104 to safely and surely access the entrance of the desired nasal sinus while the situation in the path from the external nostril E to the nasal sinus is directly recognized by the observation image. Thus, by using the insertion device assembly 12, it is possible to directly observe and treat the symptoms of each part in the nasal sinus. In this instance, if necessary, the interlock portion 200 can be used to move the distal end 106a of the sheath 106 and the distal end 252a of the insertion portion 242 of the endoscope 108 so that their positions correspond to or come closer to each other. Thus, when the sheath 106 is used to perform suction or administer a chemical, the distal end 252a of the insertion portion 242 of the endoscope 108 is temporarily evacuated to the straight pipe 174, and the distal end 252a of the insertion portion 242 of the endoscope 108 is then again brought into the nasal sinus. In this case as well, the distal end 106a of the sheath 106 is temporarily evacuated to the straight pipe 174, and the interlock portion 200 can be used so that the distal end 106a of the sheath 106 and the distal end 252a of the insertion portion 242 of the endoscope 108 pass through the bent portion 172c of the bent pipe 172 together.

In the treatment using the endoscopic treatment system 10 according to this embodiment, the insertion portion 242 of the endoscope 108 is flexible. Thus, when the distal end 252a of the insertion portion 242 of the endoscope 108 is introduced into the nasal sinus, it is not necessary to cut the entrance to the nasal sinus, crush the part around the entrance, or enlarge the entrance. The distal end portion 172a of the guide pipe 104 only holds the position relative to the entrance of the nasal sinus. Therefore, when a treatment using the treatment system 10 according to this embodiment is conducted, it is possible to significantly reduce invasive damage to the patient.

The insertion device assembly 12 according to this embodiment directly inserts the distal end 252a of the insertion portion 242 of the endoscope 108 into the nasal sinus through the distal end 104a of the guide pipe 104. Thus, the user can easily recognize the situation in the nasal sinuses. Therefore, it is possible to visually diagnose the condition of the inflammation of the mucous membrane in the nasal sinuses.

As shown in FIG. 11, it is preferable that the distal end 252a of the insertion portion 242 of the endoscope 108 slightly protrudes from the distal end 106a of the sheath 106 when the interlock portion 200 is disposed relative to the first and second operation elements 116 and 118. The interlock portion 200 is adjusted so that the distal hard portion 252 of the insertion portion 242 of the endoscope 108 protrudes from the distal end 106a of the sheath 106 by a distance shorter than at least the length of the distal hard portion 252 when the distal end 106a of the sheath 106 and the distal end 252a of the insertion portion 242 of the endoscope 108 pass through the bent portion 172c of the bent pipe 172 of the guide pipe 104. In this instance, the sheath 106 maintains the boundary between the proximal end of the distal hard portion 252 and the distal end of the flexible pipe 254. The distal end 252a of the insertion portion 242 of the endoscope 108 becomes thinner than the distal end 106a of the sheath 106. Thus, the distal end 252a of the insertion portion 242 of the endoscope 108 is thinner than when the distal end 104a of the guide pipe 104 and the distal end 252a of the insertion portion 242 of the endoscope 108 simultaneously pass through the bent pipe 172 of the guide pipe 104 or when the distal end 104a of the guide pipe 104 passes first. The resilience of the sheath 106 is difficult to be applied to the distal hard portion 252 of the insertion portion 242 of the endoscope 108. Thus, when the distal end 252a of the insertion portion 242 of the endoscope 108 passes through the bent pipe 172 of the guide pipe 104, the part including the distal hard portion 252 of the insertion portion 242 of the endoscope 108 can bend less than in the state shown in FIG. 8B.

(Modification)

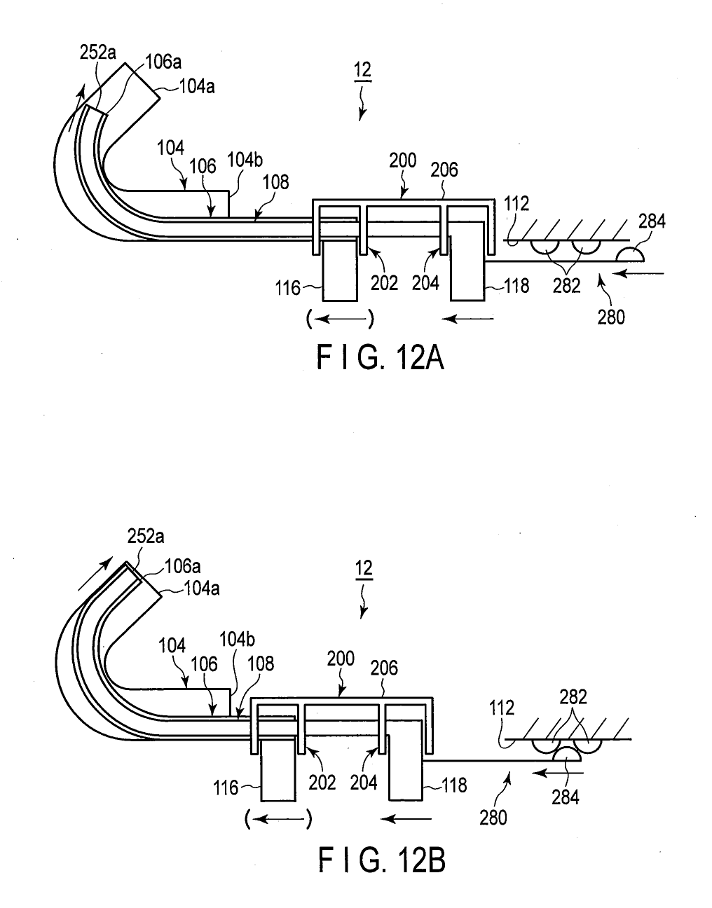

Now, a modification of the insertion device assembly 12 is described with reference to FIG. 12A and FIG. 12B.

As shown in FIG. 12A and FIG. 12B, the insertion device assembly 12 includes a click mechanism 280. The click mechanism 280 is formed by the handle unit 102 and the second operation element 118 which moves the endoscope 108. The click mechanism 280 includes a fixed-side click portion 282 provided in the main body 112 of the handle unit 102, and a movable-side click portion 284 provided in the second operation element 118. When the interlock portion 200 is attached to the first and second operation elements 116 and 118, the movable-side click portion 284 moves together with the interlock portion 200.

The click mechanism 280 allows the movable-side click portion 284 to engage with the fixed-side click portion 282 immediately after the distal end 106a of the sheath 106 and the distal end 252a of the insertion portion 242 of the endoscope 108 have passed through the bent pipe 172 of the guide pipe 104. Thus, it is possible to judge the click feeling of the click mechanism 280 that reaches the hand through the second operation element 118 and/or the interlock portion 200.

If the interlock portion 200 is moved forward relative to the handle unit 102, the distal end 106a of the sheath 106 and the distal end 252a of the insertion portion 242 of the endoscope 108 pass through the bent portion 172c of the bent pipe 172 of the guide pipe 104. Thus, the click mechanism 280 is engaged with the click feeling immediately after the distal end 106a of the sheath 106 and the distal end 252a of the insertion portion 242 of the endoscope 108 have passed through the bent portion 172c. That is, the movable-side click portion 284 engages with the fixed-side click portion 282 and generates a click feeling and a click, and the click feeling is transmitted to the hand of the user by, for example, the second operation element 118 and/or the interlock portion 200. When the click feeling is transmitted to the hand of the user in this way, the user can recognize that the distal end 106a of the sheath 106 and the distal end 252a of the insertion portion 242 of the endoscope 108 are present between the bent portion 172c of the bent pipe 172 of the guide pipe 104 and the distal opening 104a, without visually checking the monitor 16. Therefore, the user can recognize that the detachment of the interlock portion 200 from the handle unit 102 is permitted.

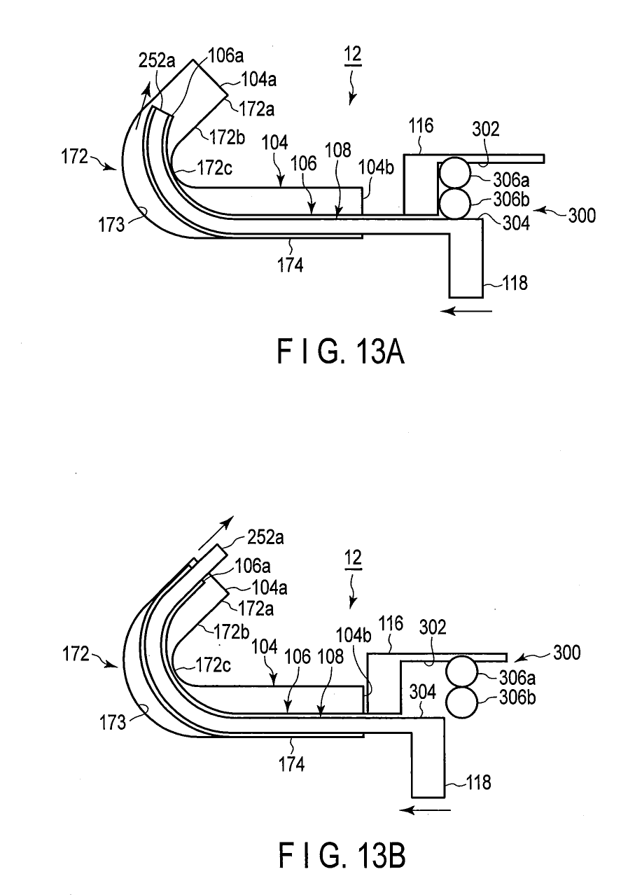

Next, the second embodiment is described with reference to FIG. 13A and FIG. 13B. This embodiment is a modification of the first embodiment including the modification described above, and the same components as the components described in the first embodiment or components having the same functions are provided with the same signs wherever possible, and are not described here.

As shown in FIG. 13A and FIG. 13B, an interlock portion 300 is provided between the first operation element 116 which moves the sheath 106 and the second operation element 118 which moves the endoscope 108 in the handle unit 102. Here, as schematically shown in FIG. 13B, the first and second operation elements 116 and 118 of the handle unit 102 can be disposed so that the proximal end of the first operation element 116 is closer to the proximal side than the proximal end of the second operation element 118, in contrast to the configuration described in the first embodiment.

As shown in FIG. 13A and FIG. 13B, the interlock portion 300 includes a first rack 302 provided in the first operation element 116, a second rack 304 provided in the second operation element 118, and first and second gears 306a and 306b. The first rack 302 has teeth formed on the surface of the first operation element 116. The second rack 304 has teeth formed on the surface of the second operation element 118. The first operation element 116 holds the first and second gears 306a and 306b.

As shown in FIG. 13A, the first gear 306a is engaged with the first rack 302 of the first operation element 116, the second gear 306b is engaged with the second rack 304 of the second operation element 118, and the first and second gears 306a and 306b are engaged with each other. For example, if the second operation element 118 is moved forward, the first gear 306a is rotated in response to the rotation of the second gear 306b by the second rack 304 of the second operation element 118. Thus, the first rack 302, that is, the first operation element 116 is moved in response to the rotation of the first gear 306a. In this case, the first operation element 116 moves forward in accordance with the forward movement of the second operation element 118. Similarly, the first operation element 116 moves backward in accordance with the backward movement of the second operation element 118.

The interlock portion 300 keeps the sheath 106 and the insertion portion 242 of the endoscope 108 moving in response to each other until the distal end 106a of the sheath 106 and the distal end 252a of the insertion portion 242 of the endoscope 108 pass through the bent portion 172c of the bent pipe 172 of the guide pipe 104. As shown in FIG. 13B, the interlock portion 300 disengages the interlock state of the first operation element 116 and the second operation element 118 after the distal end 106a of the sheath 106 and the distal end 252a of the insertion portion 242 of the endoscope 108 have passed through the bent portion 172c of the bent pipe 172 of the guide pipe 104. In this instance, the second operation element 118 can be moved forward relative to the first operation element 116. That is, the interlock portion 300 can cancel the interlock state of the sheath 106 and the insertion portion 242 of the endoscope 108 when the distal end 106a of the sheath 106 and the distal end 252a of the insertion portion 242 of the endoscope 108 have passed through the bent portion 172c of the bent pipe 172 of the guide pipe 104 and are located between the bent portion 172c and the distal end 104a of the guide pipe 104.

If the second operation element 118 is again moved backward and again interlocked with the first operation element 116, the first and second operation elements 116 and 118 move together.

(First Modification)

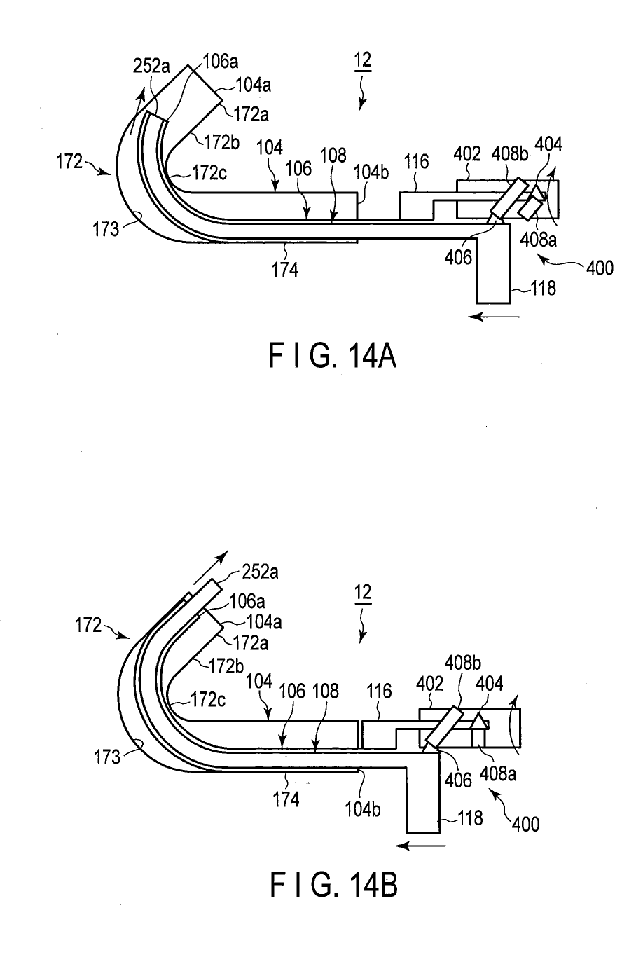

A first modification of the second embodiment is described with reference to FIG. 14A and FIG. 14B.

As shown in FIG. 14A and FIG. 14B, an interlock portion 400 is provided between the first operation element 116 which moves the sheath 106 and the second operation element 118 which moves the endoscope 108 in the handle unit 102.

The interlock portion 400 is formed as a cam mechanism here. The interlock portion 400 includes a cam cylinder 402, a first cam pin 404 provided in the first operation element 116, and a second cam pin 406 provided in the second operation element 118. The cam cylinder 402 has a first cam groove 408a which is meshed with the first cam pin 404, and a second cam groove 408b which is meshed with the second cam pin 406. The first and second cam grooves 408a and 408b may be integral, or may be suitably separated from each other.

As shown in FIG. 14A, for example, if the second operation element 118 is moved forward, the cam cylinder 402 is rotated by the second cam pin 406 of the second operation element 118 via the second cam groove 408b. In this instance, the second cam pin 406 moves forward relative to the cam cylinder 402. Due to the rotation of the cam cylinder 402, the first cam pin 404 of the first operation element 116 moves forward. Thus, the first operation element 116 is moved in response to the rotation of the cam cylinder 402. In this case, the first operation element 116 moves forward in accordance with the forward movement of the second operation element 118. Similarly, the first operation element 116 moves backward in accordance with the backward movement of the second operation element 118.

The interlock portion 400 keeps the sheath 106 and the insertion portion 242 of the endoscope 108 simultaneously moving back and forth in response to each other until the distal end 106a of the sheath 106 and the distal end 252a of the insertion portion 242 of the endoscope 108 pass through the bent portion 172c of the bent pipe 172 of the guide pipe 104. As shown in FIG. 14B, in the interlock portion 400, the first cam groove 408a of the first operation element 116 no longer has a lift after the distal end 106a of the sheath 106 and the distal end 252a of the insertion portion 242 of the endoscope 108 have passed through the bent portion 172c of the bent pipe 172 of the guide pipe 104. In this instance, the second operation element 118 can be moved forward relative to the first operation element 116. That is, the interlock portion 400 can cancel the state in which the sheath 106 and the insertion portion 242 of the endoscope 108 simultaneously move back and forth when the distal end 106a of the sheath 106 and the distal end 252a of the insertion portion 242 of the endoscope 108 have passed through the bent portion 172c of the bent pipe 172 of the guide pipe 104 and are located between the bent portion 172c of the bent pipe 172 and the distal end 104a of the guide pipe 104.

If the second operation element 118 is moved backward and then again interlocked with the first operation element 116, the first and second operation elements 116 and 118 move together.

(Second Modification)

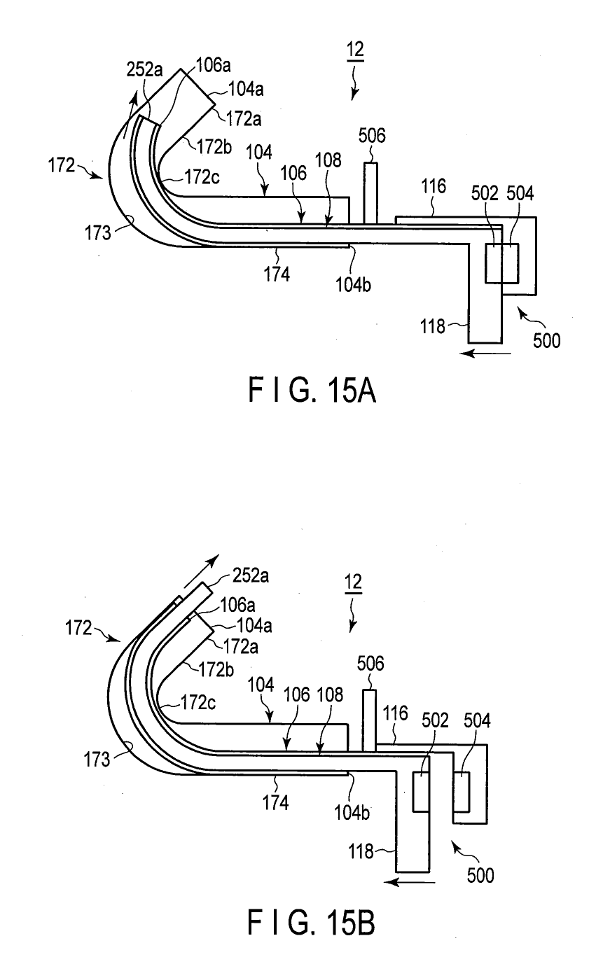

A second modification of the second embodiment is described with reference to FIG. 15A and FIG. 15B.

As shown in FIG. 15A and FIG. 15B, an interlock portion (synchronizing portion) 500 is provided between the first operation element 116 which moves the sheath 106 and the second operation element 118 which moves the endoscope 108 in the handle unit 102.

The interlock portion 500 includes a first magnet 502 provided in the first operation element 116, and a second magnet 504 provided in the second operation element 118. The first and second magnets 502 and 504 exert an attraction force on each other. The interlock portion 500 has a stopper 506. The stopper 506 regulates the forward movement of the first operation element 116. The stopper 506 is provided in, for example, the handle unit 102.

As shown in FIG. 15A, for example, if the second operation element 118 is moved forward, the first and second magnets 502 and 504 also move the first operation element 116 together by their attraction force. In this case, the first operation element 116 moves forward in accordance with the forward movement of the second operation element 118. Similarly, the first operation element 116 moves backward in accordance with the backward movement of the second operation element 118.

The interlock portion 500 keeps the sheath 106 and the insertion portion 242 of the endoscope 108 moving together until the distal end 106a of the sheath 106 and the distal end 252a of the insertion portion 242 of the endoscope 108 pass through the bent portion 172c of the bent pipe 172 of the guide pipe 104. As shown in FIG. 15B, in the interlock portion 500, the first operation element 116 abuts on the stopper 506 after the distal end 106a of the sheath 106 and the distal end 252a of the insertion portion 242 of the endoscope 108 have passed through the bent portion 172c of the bent pipe 172 of the guide pipe 104. Thus, if the second operation element 118 is moved forward, the interlock state of the first operation element 116 and the second operation element 118 is canceled. In this instance, the second operation element 118 can be moved forward relative to the first operation element 116. That is, the interlock portion 500 can cancel the interlock of the sheath 106 and the insertion portion 242 of the endoscope 108 when the distal end 106a of the sheath 106 and the distal end 252a of the insertion portion 242 of the endoscope 108 have passed through the bent portion 172c of the bent pipe 172 of the guide pipe 104 and are located between the bent portion 172c of the bent pipe 172 and the distal end 104a of the guide pipe 104.

If the second operation element 118 is moved backward and then again interlocked with the first operation element 116, the first and second operation elements 116 and 118 move together.

Therefore, the interlock portion 500 can switch between the interlock state in which the first operation element 116 and the second operation element 118 are moved along the longitudinal direction of the sheath 106 and the insertion portion 242 of the endoscope 108 so that the first operation element 116 and the second operation element 118 are interlocked, and the non-interlock state in which the first operation element 116 and the second operation element 118 can be independently moved.

Although the first and second magnets 502 and 504 are used in the example described here, it is also preferable to use, for example, a hook-and-loop fastener instead of the magnet.

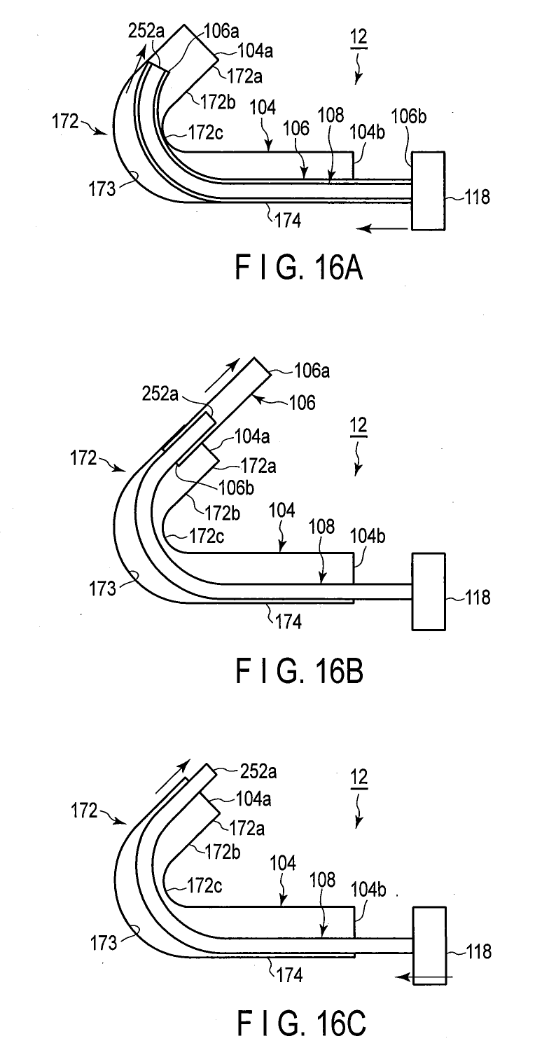

Next, the third embodiment is described with reference to FIG. 16A to FIG. 16C. This embodiment is a modification of the first and second embodiments including the modifications described above, and the same components as the components described in the first and second embodiments or components having the same functions are provided with the same signs as much as possible, and are not described here.

The sheath 106 according to this embodiment is different from the sheath 106 described in the first and second embodiments. It is preferable that the sheath 106 according to this embodiment is only formed by the sheath body 152 and is not provided with the sheath holder 154 and the inner pipe 156 shown in FIG. 6. Thus, the handle unit 102 has the operation element 118 which moves the endoscope 108, but does not need the first operation element 116 (see FIG. 8A) which moves the sheath 106.

As shown in FIG. 16A and FIG. 16B, the proximal side opening 106b of the sheath 106 is inseparably coupled to the second operation element 118 of the insertion portion 242 of the endoscope 108. That is, the proximal side opening 106b of the sheath 106 and the second operation element 118 of the insertion portion 242 of the endoscope 108 are interlocked when moving forward. That is, the sheath 106 moves forward together with the forward movement of the second operation element 118 of the endoscope 108. On the other hand, if the second operation element 118 of the endoscope 108 is moved backward along the handle unit 102, the sheath 106 maintains its position due to the similarity in function to that of the O-ring 120a described above (see FIG. 5A).

As shown in FIG. 16A, if the second operation element 118 is moved forward relative to the handle unit 102, the sheath 106 moves forward together with the insertion portion 242 of the endoscope 108. The sheath 106 and the insertion portion 242 of the endoscope 108 are kept moving together until the distal end 106a of the sheath 106 and the distal end 252a of the insertion portion 242 of the endoscope 108 pass through the bent pipe 172 of the guide pipe 104 and further pass through the distal opening 104a of the guide pipe 104.

The handle unit 102 maintains the position of the second operation element 118 in the state in which the distal end 106a of the sheath 106 and the distal end 252a of the insertion portion 242 of the endoscope 108 have passed through the distal opening 104a of the guide pipe 104. As shown in FIG. 16B, the sheath 106 is then pulled off through the distal opening 104a of the guide pipe 104 while the distal end 252a of the insertion portion 242 of the endoscope 108 is protruding from the distal opening 104a of the guide pipe 104.

As shown in FIG. 16C, the second operation element 118 of the endoscope 108 is moved relative to the handle unit 102. Thus, the distal end portion 172a of the guide pipe 104 is put into the entrance G of the maxillary sinus M of the nasal sinuses while the distal end 252a of the insertion portion 242 of the endoscope 108 is slightly retracted relative to the distal opening 104a of the guide pipe 104.

In the first to third embodiments described above, the distal end 106a of the sheath 106 and the distal end 252a of the insertion portion 242 of the endoscope 108 can be put through the bent portion 172c of the guide pipe 104, and the distal end 252a of the insertion portion 242 of the endoscope 108 can be protruded from the distal opening 104a of the guide pipe 104. To perform such an operation, the first and second operation elements 116 and 118 are used in the examples described in the first and second embodiments, and one second operation element 118 is used in the example described in the third embodiment. According to the insertion device assembly 12 in the first to third embodiments described above, it is possible to surely access a desired position in the nasal sinus and then certainly observe the inside of the nasal sinus.

Although the distal end portion 172a of the guide pipe 104 of the insertion device assembly 12 is disposed in the entrance G of the maxillary sinus M as an example of the nasal sinus in the examples described, the nasal sinus to be treated is not limited to the maxillary sinus M.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An insertion device assembly for nasal sinuses comprising:
a guide pipe main body;
a guide pipe bent portion provided on the distal side of the guide pipe main body;
a guide pipe distal opening provided on the distal side of the guide pipe bent portion;
a sheath which is inserted into the guide pipe bent portion and the guide pipe distal opening from a proximal side opening of the guide pipe main body through the guide pipe main body to be movable back and forth;
a sheath distal opening provided at the distal side of the sheath;
an insertion portion of an endoscope which is inserted through the sheath to be movable back and forth; and
an endoscope operation portion which is configured to pass the sheath distal opening and the distal end of the insertion portion of the endoscope through the guide pipe bent portion and which is configured to protrude the distal end of the insertion portion of the endoscope from the guide pipe distal opening.

2. The insertion device assembly according to claim 1, further comprising:
a sheath operation portion which is configured to dispose the sheath distal opening closer to the distal side than the guide pipe bent portion and which is configured to protrude the sheath distal opening from the guide pipe distal opening; and
an interlock portion which interlocks the sheath operation portion and the endoscope operation portion.

3. The insertion device assembly according to claim 2, wherein the interlock portion is configured to cancel the interlock of the sheath and the insertion portion of the endoscope when the sheath distal opening and the distal end of the insertion portion of the endoscope have passed through the guide pipe bent portion and are located between the guide pipe bent portion and the guide pipe distal opening.

4. The insertion device assembly according to claim 2, wherein the interlock portion includes a synchronizing portion which synchronizes the operations of the sheath operation portion and the endoscope operation portion.

5. The insertion device assembly according to claim 2, wherein the interlock portion includes racks respectively provided in the sheath operation portion and the endoscope operation portion, and a gear provided between the racks.

6. The insertion device assembly according to claim 2, wherein the interlock portion includes a cam mechanism provided between the sheath operation portion and the endoscope operation portion.

7. The insertion device assembly according to claim 2, wherein the interlock portion is configured to move the sheath operation portion and the endoscope operation portion along the longitudinal direction of the sheath and the insertion portion of the endoscope to switch between an interlock state and a non-interlock state.

8. The insertion device assembly according to claim 2, comprising:
a click mechanism which generates a click feeling when the sheath distal opening and the distal end of the insertion portion of the endoscope have passed through the guide pipe bent portion and come close to the guide pipe distal opening.

9. The insertion device assembly according to claim 2, wherein
the insertion portion of the endoscope includes a distal hard portion located at the distal end, and a flexible pipe provided on the proximal side of the distal hard portion, and
the interlock portion protrudes the distal hard portion from the sheath distal opening by a distance shorter than at least the length of the distal hard portion when the sheath distal opening and the distal end of the insertion portion of the endoscope pass through the guide pipe bent portion.

10. The insertion device assembly according to claim 1, wherein the sheath is configured to be pulled out of the guide pipe distal opening when the sheath distal opening and the distal end of the insertion portion of the endoscope have passed through the guide pipe bent portion and the guide pipe distal opening.

11. The insertion device assembly according to claim 1, wherein the guide pipe bent portion is bent at about 110° relative to the guide pipe main body.

12. The insertion device assembly according to claim 1, wherein the guide pipe bent portion is bent at a bending radius of 5 mm to 20 mm at the distal end of the guide pipe main body.
